SEQUENCE LISTING (37 sequences)

SEQ ID NO: 1 - protein, 410 aa, Homo sapiens
MAFLGLFSLLVLQSMATGATFPEEAIADLSVNMYNRLRATGEDENILFSPLSIALAMGMMELGAQGSTQKEIRHSMGYDSLKNGEEFSFLKEFSNMVTAKESQYVMKIANSLFVQNGFHVNEEFLQMMKKYFNAAVNHVDFSQNVAVANYINKWVENNTNNLVKDLVSPRDFDAATYLALINAVYFKGNWKSQFRPENTRTFSFTKDDESEVQIPMMYQQGEFYYGEFSDGSNEAGGIYQVLEIPYEGDEISMMLVLSRQEVPLATLEPLVKAQLVEEWANSVKKQKVEVYLPRFTVEQEIDLKDVLKALGITEIFIKDANLTGLSDNKEIFLSKAIHKSFLEVNEEGSEAAAVSGMIAISRMAVLYPQVIVDHPFFFLIRNRRTGTILFMGRVMHPETMNTSGHDFEEL

SEQ ID NO: 2 - protein, 410 aa, Artificial Sequence
  Mutant neuroserpin
MAFLGLFSLLVLQSMATGATFPEEAIADLSVNMYNRLRATGEDENILFSPLSIALAMGMMELGAQGSTQKEIRHSMGYDSLKNGEEFSFLKEFSNMVTAKESQYVMKIANSLFVQNGFHVNEEFLQMMKKYFNAAVNHVDFSQNVAVANYINKWVENNTNNLVKDLVSPRDFDAATYLALINAVYFKGNWKSQFRPENTRTFSFTKDDESEVQIPMMYQQGEFYYGEFSDGSNEAGGIYQVLEIPYEGDEISMMLVLSRQEVPLATLEPLVKAQLVEEWANSVKKQKVEVYLPRFTVEQEIDLKDVLKALGITEIFIKDANLTGLSDNKEIFLSKAIHKSFLEVNEEGSEAAAVSGMIAISRRAVLYPQVIVDHPFFFLIRNRRTGTILFMGRVMHPETMNTSGHDFEEL

SEQ ID NO: 3 - protein, 410 aa, Pan troglodytes
MAFLGLFSLLVLQSMATAATFPEEAIADLSVNMYNRLRATGEDENILFSPLSIALAMGMMELGAQGSTQKEIRHSMGYDSLKNGEEFSFLKEFSNMVTAKESQYVMKIANSLFVQNGFHVNEEFLQMMKKYFNAEVNHVDFSQNVAVANYINKWVENNTNNLVKDLVSPRDFDAATYLALINAVYFKGNWKSQFRPENTRTFSFTKDDESEVQIPMMYQQGEFYYGEFSDGSNEAGGIYQVLEIPYEGDEISMMLVLSRQEVPLATLEPLVKAQLIEEWANSVKKQKVEVYLPRFTVEQEIDLKDVLKALGITEIFIKDANLTGLSDNKEIFLSKAIHKSFLEVNEEGSEAAAVSGMIAISRMAVLYPQVIVDHPFFFLIRNRRTGTILFMGRVMHPETMNTSGHDFEEL

SEQ ID NO: 4 - protein, 410 aa, Mus musculus
MTYLELLALLALQSVVTGATFPDETITEWSVNMYNHLRGTGEDENILFSPLSIALAMGMMELGAQGSTRKEIRHSMGYEGLKGGEEFSFLRDFSNMASAEENQYVMKLANSLFVQNGFHVNEEFLQMLKMYFNAEVNHVDFSQNVAVANSINKWVENYTNSLLKDLVSPEDFDGVTNLALINAVYFKGNWKSQFRPENTRTFSFTKDDESEVQIPMMYQQGEFYYGEFSDGSNEAGGIYQVLEIPYEGDEISMMLALSRQEVPLATLEPLLKAQLIEEWANSVKKQKVEVYLPRFTVEQEIDLKDILKALGVTEIFIKDANLTAMSDKKELFLSKAVHKSCIEVNEEGSEAAAASGMIAISRMAVLYPQVIVDHPFLYLIRNRKSGIILFMGRVMNPETMNTSGHDFEEL

SEQ ID NO: 5 - protein, 410 aa, Rattus rattus
MAYLGLLSLVAMQSLVTGAAFPDETIAEWSVNVYNHLRATGEDENILFSPLSIALAMGVMELGAQGSTLKEIRHSMGYESLKSGEEFSFLRDFSSMVSAEEGQYVMKIANSLFVQNGFHINEEFLQMMKMYFNAEVNHVDFSENVAVANYINKWVENYTNSLLKDLVSPGDFDAVTQLALINAVYFKGNWKSQFRPENTRTFSFTKDDESEVQIPMMYQQGEFYYGEFSDGSNEAGGIYQVLEIPYEGDEISMMLVLSRQEVPLATLEPLLKPQLIEEWANSVKKQKVEVYLPRFTVEQEIDLKDILKALGVTEIFIKDANLTAMSDKKELFLSKAVHKSFIEVNEEGSEAAVASGMIAISRMAVLFPQVIVDHPFLFLIKNRKTGTILFMGRVMHPETMNTSGHDFEEL

SEQ ID NO: 6 - protein, 410 aa, Cattle
  misc_feature (250)..(250): Xaa can be any naturally occurring amino acid
MAFLGLFSLLVLQSLALGTTFTDETIAELSVNMYNHLRATGEDENILFSPLSVTLAMGMLELGAQGSTLKEIRHSMGYDSLKNGEEFSFLKDLSHMVTAEESQYVMKIANSLFVQNGFHISEEFLQMIKKYFNAEVNHVDFSQNVAVANYINKWVENNTNSLLKDLVSPRDFDAVTHLALINAVYFKGSWKSQFRPENTRTFSFTKDDESEVQIPMMYQQGEFYYGEFSDGSNEAGGIYQVLEIPYEGDXISMMLVLSRQEVPLATLEPLVKAQLIEEWANSVKKQKVEVYLPRFTVEQEIDLKDVLKALGVTEVFIKNANLTALSDNKEIFLSKAIHKCFIEVNEEGSEAAAASGMIAISRMAVLYPQVIVDHPFFFLIRNRRTGTILFMGRVMHPETMNASGHDFEEL

SEQ ID NO: 7 - protein, 410 aa, Pig
MAFLGFFSLLVLQSLALGTTFTDETITEFSVNMYNHLRATGEDENILFSPLSVTLAMGMLELGAQGSTLKEIRHSMGYESLKSGEEFSFLKDLSHMVTAEESQYVMKIANSLFVQNGFHVNDEFLQMLKKYFNAEVNHVDFSQNVAVANYINKWVENNTNSLLKDLLSPTDFDAVTHLALINAVYFKGNWKSQFRPENTRTFSFTKDDESEVQIPMMYQQGEFYYGEFSDGSNEAGGIYQVLEIPYEGDEISMMLVLSRQEVPLATLEPLVKAQLLEEWANSVKKQKVEVYLPRFTVEQEVDLKNVLKALGITEVFIKNANLTALSDNKEIFLSKAIHKSFIEVNEEGSEAAAASGMIAISRMAVLYPQVIVDHPFFFLIRNRRSGTILFMGRVMHPETMNTSGHDFEEL

SEQ ID NO: 8 - protein, 410 aa, Rhesus monkey
MALLGLFSLLVLQSMTVGATFPEEAIADLSVNMYNRLRATGEDENIIFSPLSIALAMGMMELGAQGSTQKEIRHSMGYDSLKNGEEFSFLKEFSNVVTAKESQYVMKIANSLFVQNGFHINEEFLQMMKKYFNAEVNHVDFSQNVAVANYINKWVENNTNNLVKDLVSPRDFDAATYLALINAVYFKGNWKSQFRPENTRTFSFTKDDESEVQIPMMYQQGEFYYGEFSDGSNEAGGIYQVLEIPYEGDEISMMLVLSRQEVPLATLEPLVKAQLVEEWANSVKKQKVEVYLPRFTVEQEIDLKDVLKALGITEIFIKDANLTALSDNKEIFLSKAIHKSFLEVNEEGSEAAAVSGMIAISRMAVLYPQVIVDHPFFFLIRNRRTGTILFMGRVMHPETMNTSGHDFEEL

SEQ ID NO: 9 - protein, 410 aa, Chicken
MYFLGLLSLLVLPSKAFKTNFPDETIAELSVNVYNQLRAAREDENILFCPLSIAIAMGMIELGAHGTTLKEIRHSLGFDSLKNGEEFTFLKDLSDMATTEESHYVLNMANSLYVQNGFHVSEKFLQLVKKYFKAEVENIDFSQSAAVATHINKWVENHTNNMIKDFVSSRDFSALTHLVLINAIYFKGNWKSQFRPENTRTFSFTKDDETEVQIPMMYQQGEFYYGEFSDGSNEAGGIYQVLEIPYEGDEISMMIVLSRQEVPLVTLEPLVKASLINEWANSVKKQKVEVYLPRFTVEQEIDLKDVLKGLGITEVFSRSADLTAMSDNKELYLAKAFHKAFLEVNEEGSEAAAASGMIAISRMAVLYPQVIVDHPFFFLVRNRRTGTVLFMGRVMHPEAMNTSGHDFEEL

SEQ ID NO: 10 - protein, 410 aa, Xenopus laevis
MHHFILLSLIVMQALVFGTSVHDETVNEFSVKVYHELRATKDDENIIFSPLSTAIALGMVELGARGSSLKEIRHVLGYDKLKNGEEFSLLKDLSSMLTAQEKHYVLSIANSLYLQNGFHISDKFIQLMKKYFKAEVENVDFSQGSTVANHINLWVENHTNNRIRDLFTADDFNNLTKLVLVNAMYFKGNWKSQFRPENTRTFSFTKDDESEVQIPMMYQKGEFYYGEFTDGSNEAGGVYQVLELPYEGDEISLIIVLSRQEVPLATIEPLLKAHLIEEWAKSVKKQKVEVYLPRFKVEEVVNLKEVLMRLGITKIFSGEADLSAISDSKDLFVAKAVHKSFLEINEEGSEAAASSGMIANSRMAVLYPQVIVDHPFFFVIRNRKTGSVLFMGRVMHPETLTTTGHDFEEL

SEQ ID NO: 11 - protein, 410 aa, Sheep
MAFLGLFSLLVLQSLALGTTFTDETIAELSVNMYNHLRATGEDENILFSPLSVTLAMGMLELGAQGSTLKEIRHSMGYDSLRNGEEFSFLKDLSHMVTAEESQYVMKIANSLFVQNGFHISEEFLQMIKKYFNAEINHVDFSQNVAVANYINKWVENNTNSLLKDLVSPRDFDAVTHLALINAVYFKGSWKSQFRPENTRTFSFTKDDESEVQIPMMYQQGEFYYGEFSDGSNEAGGIYQVLEIPYEGDAISMMLVLSRQEVPLATLEPLVKAQLIEEWANSVKKQKVEVYLPRFTVEQEIDLKDVLKALGVTEVFIKNANLTALSDNKEIFLSKAIHKCFIEVNEEGSEAAAASGMIAISRMAVLYPQVIVDHPFFFLIRNRRTGTILFMGRVMHPETMNASGHDFEEL

SEQ ID NO: 12 - protein, 411 aa, Chiloscyllium punctatum
MASLLGFLIVSIVKYVMVDAGIRDGITADFSVKMYHYLRSLEKDGNIFYSPFSIANALGMVELGAGSTTLEQIQHVMGYHYLQKGMEFDLLRDLSQSMKADSQQYVMKLVNSLFVEKGFQLSDQFLQMLKKYFGASVESIDFSQPSFVADHINEWVQNQTNNKIPNLLSPKDLDALTRLVLVNAIYFKGNWKSEFRGENTHTFQFTKDDESEIQIAMMYQQGDFNYGEFSDGSNEAGGVYQVLEMPYIGDEMSMMIVLPRQEVQLATLEPLIKTQLINEWASSVKKQKVEVYLPRFKMTQNIDLKEALAGLGISEMFGVEADLSAMTEKKDLHISKAIHTSFIEVTEEGSEAAAASEMIAVSRMMVLTPQIIADHPFFFLIRNQRTGTILFMGRVMHPENIHPNEHDFEAL

SEQ ID NO: 13 - DNA, 1233 bp, Homo sapiens
atggctttccttggactcttctctttgctggttctgcaaagtatggctacaggggccactttccctgaggaagccattgctgacttgtcagtgaatatgtataatcgtcttagagccactggtgaagatgaaaatattctcttctctccattgagtattgctcttgcaatgggaatgatggaacttggggcccaaggatctacccaggaagaaatccgccactcaatgggatatgacagcctaaaaaatggtgaagaattttctttcttgaaggagttttcaaacatggtaactgctaaagagagccaatatgtgatgaaaattgccaattccttgtttgtgcaaaatggatttcatgtcaatgaggagtttttgcaaatgatgaaaaaatattttaatgcagcagtaaatcatgtggacttcagtcaaaatgtagccgtggccaactacatcaataagtgggtggagaataacacaaacaatctggtgaaagatttggtatccccaagggattttgatgctgccacttatctggccctcattaatgctgtctatttcaaggggaactggaagtcgcagtttaggcctgaaaatactagaaccttttctttcactaaagatgatgaaagtgaagtccaaattccaatgatgtatcagcaaggagaattttattatggggaatttagtgatggctccaatgaagctggtggtatctaccaagtcctagaaataccatatgaaggagatgaaataagcatgatgctggtgctgtccagacaggaagttcctcttgctactctggagccattagtcaaagcacagctggttgaagaatgggcaaactctgtgaagaagcaaaaagtagaagtatacctgcccaggttcacagtggaacaggaaattgatttaaaagatgttttgaaggctcttggaataactgaaattttcatcaaagatgcaaatttgacaggcctctatgataataaggagatttttctttccaaagcaattcacaagtccttcctagaggttaatgaagaaggctcagaagctgctgctgtctcaggaatgattgcaattagtaggatggctgtgctgtatcctcaagttattgtcgaccatccatttttctttcttatcagaaacaggagaactggtacaattctattcatgggacgagtcatgcatcctgaaacaatgaacacaagtggacatgatttcgaagaactttaa

SEQ ID NO: 14 - protein, 10 aa, Artificial Sequence
  mutant portion of reactive centre loop of neuroserpin
  MISC_FEATURE (4)..(4): Xaa is I, V or N
  MISC_FEATURE (7)..(7): Xaa is R, S, T, F, N, E, K or D
  MISC_FEATURE (8)..(8): Xaa is M or A
MIAXSRXXVL

SEQ ID NO: 15 - protein, 10 aa, Artificial Sequence
  mutant  reactive centre loop sequence from neuroserpin
  MISC_FEATURE (7)..(7): Xaa is R, S, T, F, N, E, K or D
MIAISRXAVL

SEQ ID NO: 16 - protein, 10 aa, Artificial Sequence
  mutant reactive centre loop sequence of neuroserpin
MIAISRRAVL

SEQ ID NO: 17 - protein, 9 aa, Artificial Sequence
  mutant reactive centre loop seuqnce of neuroserpin
IAISRMAVL

SEQ ID NO: 18 - protein, 13 aa, Artificial Sequence
  mutant reactive centre loop sequence of neuropserpin
IAISRMAVLYPQV

SEQ ID NO: 19 - protein, 9 aa, Artificial Sequence
  portion of  reactive centre loop sequence of neuroserpin
  MISC_FEATURE (3)..(3): Xaa is I, V or N
IAXSRMAVL

SEQ ID NO: 20 - protein, 11 aa, Artificial Sequence
  mutant reactive centre loop sequence os neuroserpin
  MISC_FEATURE (4)..(4): Xaa is I, V or N
  MISC_FEATURE (7)..(7): Xaa is R, S, T, F, N, E, K or D
  MISC_FEATURE (11)..(11): Xaa is Y or F
MIAXSRXAVLX

SEQ ID NO: 21 - protein, 14 aa, Artificial Sequence
  mutant reactive centre loop sequence of neuroserpin
  MISC_FEATURE (4)..(4): Xaa is I, V or N
  MISC_FEATURE (7)..(7): Xaa is R, S, T, F, N, E, K or D
  MISC_FEATURE (11)..(11): Xaa is Y or F
MIAXSRXAVLXPQV

SEQ ID NO: 22 - protein, 15 aa, Artificial Sequence
  mutant reactive centre loop sequence of neuroserpin
  MISC_FEATURE (7)..(7): Xaa is R, S, T, F, N, E, K or D
  MISC_FEATURE (11)..(11): Xaa is Y or F
MIAISRXAVLXPQVI

SEQ ID NO: 23 - protein, 17 aa, Artificial Sequence
  mutant reactive centre loop sequence of neuroserpin
  MISC_FEATURE (8)..(8): Xaa is R, S, T, F, N, E, K or D
  MISC_FEATURE (12)..(12): Xaa is Y or F
GMIAISRXAVLXPQVIV

SEQ ID NO: 24 - protein, 22 aa, Artificial Sequence
  mutant reactive centre loop sequence of neuroserpin
  MISC_FEATURE (9)..(9): Xaa is R, S, T, F, N, E, K or D
  MISC_FEATURE (13)..(13): Xaa is Y or F
SGMIAISRXAVLXPQVIVDHPF

SEQ ID NO: 25 - protein, 13 aa, Artificial Sequence
  mutant reactive centre loop sequence of neuroserpin
IAISRRAVLYPQV

SEQ ID NO: 26 - protein, 15 aa, Artificial Sequence
  mutant reactive centre loop sequence of neuroserpin
MIAISRRAVLYPQVI

SEQ ID NO: 27 - protein, 17 aa, Artificial Sequence
  mutant reactive centre loop sequence of neuroserpin
GMIAISRRAVLYPQVIV

SEQ ID NO: 28 - protein, 22 aa, Artificial Sequence
  mutant reactive centre loop sequence of neuroserpin
SGMIAISRRAVLYPQVIVDHPF

SEQ ID NO: 29 - protein, 37 aa, Artificial Sequence
  mutant neuroserpin sequence
EAAAVSGMIAISRRAVLYPQVIVDHPFFFLIRNRRTG

SEQ ID NO: 30 - protein, 52 aa, Artificial Sequence
  mutant neuroserpin sequence
LEVNEEGSEAAAVSGMIAISRRAVLYPQVIVDHPFFFLIRNRRTGTILFMGR

SEQ ID NO: 31 - protein, 67 aa, Artificial Sequence
  mutant neuroserpin sequence
SKAIHKSFLEVNEEGSEAAAVSGMIAISRRAVLYPQVIVDHPFFFLIRNRRTGTILFMGRVMHPETM

SEQ ID NO: 32 - protein, 90 aa, Artificial Sequence
  mutant neuroserpin sequence
NLTGLSDNKEIFLSKAIHKSFLEVNEEGSEAAAVSGMIAISRRAVLYPQVIVDHPFFFLIRNRRTGTILFMGRVMHPETMNTSGHDFEEL

SEQ ID NO: 33 - DNA, 1605 bp, Homo sapiens
agtttcccgggagagacgaaagcaggaacgagagcggagcggagcacagtccgccgagcacaagctccagcatcccgtcaggggttgcaggtgtgtgggaggcttgaaactgttacaatatggctttccttggactcttctctttgctggttctgcaaagtatggctacaggggccactttccctgaggaagccattgctgacttgtcagtgaatatgtataatcgtcttagagccactggtgaagatgaaaatattctcttctctccattgagtattgctcttgcaatgggaatgatggaacttggggcccaaggatctacccaggaagaaatccgccactcaatgggatatgacagcctaaaaaatggtgaagaattttctttcttgaaggagttttcaaacatggtaactgctaaagagagccaatatgtgatgaaaattgccaattccttgtttgtgcaaaatggatttcatgtcaatgaggagtttttgcaaatgatgaaaaaatattttaatgcagcagtaaatcatgtggacttcagtcaaaatgtagccgtggccaactacatcaataagtgggtggagaataacacaaacaatctggtgaaagatttggtatccccaagggattttgatgctgccacttatctggccctcattaatgctgtctatttcaaggggaactggaagtcgcagtttaggcctgaaaatactagaaccttttctttcactaaagatgatgaaagtgaagtccaaattccaatgatgtatcagcaaggagaattttattatggggaatttagtgatggctccaatgaagctggtggtatctaccaagtcctagaaataccatatgaaggagatgaaataagcatgatgctggtgctgtccagacaggaagttcctcttgctactctggagccattagtcaaagcacagctggttgaagaatgggcaaactctgtgaagaagcaaaaagtagaagtatacctgcccaggttcacagtggaacaggaaattgatttaaaagatgttttgaaggctcttggaataactgaaattttcatcaaagatgcaaatttgacaggcctctatgataataaggagatttttctttccaaagcaattcacaagtccttcctagaggttaatgaagaaggctcagaagctgctgctgtctcaggaatgattgcaattagtaggatggctgtgctgtatcctcaagttattgtcgaccatccatttttctttcttatcagaaacaggagaactggtacaattctattcatgggacgagtcatgcatcctgaaacaatgaacacaagtggacatgatttcgaagaactttaagttactttatttgaataacaaggaaaacagtaactaagcacattatgtttgcaactggtatatatttaggatttgtgttttacagtatatcttaagataatatttaaaatagttccagataaaaacaatatatgtaaattataagtaacttgtcaaggaatgttatcagtattaagctaatggtcctgttatgtcattgtgtttgtgtgctgttgtttaaaataaaagtacctatcgaaaaaaaaaaaaaaaa

SEQ ID NO: 34 - protein, 10 aa, Artificial Sequence
  mutant reactice centre loop sequence of neuroserpin
  MISC_FEATURE (4)..(4): Xaa is I,, V or N
  MISC_FEATURE (7)..(7): Xaa is an amino acid other than M
  MISC_FEATURE (8)..(8): Xaa is M or A
MIAXSRXXVL

SEQ ID NO: 35 - protein, 410 aa, Artificial Sequence
  mutant reactive centre loop sequence of neuropserpin
  MISC_FEATURE (363)..(363): Xaa is an amino acid other than M
MAFLGLFSLLVLQSMATGATFPEEAIADLSVNMYNRLRATGEDENILFSPLSIALAMGMMELGAQGSTQKEIRHSMGYDSLKNGEEFSFLKEFSNMVTAKESQYVMKIANSLFVQNGFHVNEEFLQMMKKYFNAAVNHVDFSQNVAVANYINKWVENNTNNLVKDLVSPRDFDAATYLALINAVYFKGNWKSQFRPENTRTFSFTKDDESEVQIPMMYQQGEFYYGEFSDGSNEAGGIYQVLEIPYEGDEISMMLVLSRQEVPLATLEPLVKAQLVEEWANSVKKQKVEVYLPRFTVEQEIDLKDVLKALGITEIFIKDANLTGLSDNKEIFLSKAIHKSFLEVNEEGSEAAAVSGMIAISRXAVLYPQVIVDHPFFFLIRNRRTGTILFMGRVMHPETMNTSGHDFEEL

SEQ ID NO: 36 - protein, 9 aa, Artificial Sequence
  Conserved concensus region from reactive centre loop
  MISC_FEATURE (3)..(3): Xaa is I, V or N
  MISC_FEATURE (7)..(7): Xaa is M or A
IAXSRMXVL

SEQ ID NO: 37 - protein, 9 aa, Artificial Sequence
  mutant neuroserpin sequence
  MISC_FEATURE (3)..(3): Xaa is I, V or N
  MISC_FEATURE (6)..(6): Xaa is an amino acid other than M
IAXSRXAVL

(19) United States
(12) Patent Application Publication
(10) Pub. No.: US 2024/0067703 A1
(43) Pub. Date: Feb. 29, 2024

(54) TREATMENT OF GLAUCOMA

(71) Applicant: MACQUARIE UNIVERSITY, North Ryde, New South Wales (AU)

(72) Inventors: Nitin Chitranshi, North Ryde (AU); Vivek Gupta, North Ryde (AU); Stuart Graham, North Ryde (AU); Rashi Rajput, North Ryde (AU)

(73) Assignee: MACQUARIE UNIVERSITY, North Ryde, New South Wales (AU)

(21) Appl. No.: 18/268,203

(22) PCT Filed: Dec. 20, 2021
(86) PCT No.: PCT/AU2021/051524

§ 371 Date: Jun. 16, 2023

(30) Foreign Application Priority Data
Dec. 21, 2020 (AU) 2020904767
Jul. 15, 2021 (AU) 2021902179

(51) Int. Cl. C07K 14/81; A61P 27/06

(52) U.S. Cl. CPC C07K 14/8121; A61P 27/06; A61K 38/00

(57) ABSTRACT

The present disclosure relates to a mutant neuroserpin protein or portion thereof, to a nucleic acid comprising nucleotide sequence which encodes a mutant neuroserpin protein or portion thereof, and to use of the nucleic acid or mutant neuroserpin protein or portion thereof for treating glaucoma and other disorders associated with elevated plasmin activity.

### FIELD

The present disclosure relates to a nucleic acid comprising nucleotide sequence which encodes a mutant neuroserpin protein or portion thereof, to a mutant neuroserpin protein or portion thereof, and to use of the nucleic acid and mutant neuroserpin protein or portion thereof for treating glaucoma and other neurodegenerative disorders associated with plasmin activity.

### BACKGROUND

Glaucoma is a neurodegenerative disease often associated with increased intraocular pressure (TOP). Glaucoma causes retinal ganglion cell (RGC) degeneration and excavation of the optic nerve head, leading to vision loss.

Several factors such as pressure induced remodelling of the lamina cribrosa, axonal compression of the RGCs, obstruction in the retrograde flow of neurotrophins to RGCs, impediments in axonal transport along the optic nerve, chronic ischemic insult and digestion of the extracellular matrix (ECM) at the optic nerve head by proteolytic activity have been suggested to play a role in the glaucoma pathology.

Increased intraocular pressure (IOP) is considered a prominent manifestation of glaucoma, and controlling IOP remains the primary means of disease management. Various medications are available for managing IOP, and these include, for example, prostaglandin analogs (such as latanoprost, bimatoprost, travoprost tafluprost, and latanoprostene bunod), beta blockers (such as Betaxolol, timolol), alpha-adrenergic agonists (Apraclonidine, Brimonidine), and combinations thereof.

Management of IOP using known methods only slows the progression of glaucoma, but does not cure the disease. Also, loss of RGCs can occur in glaucoma cases even when IOP is reduced, and some patient still get glaucoma with normal IOP.

What is needed is alternative approaches for the treatment of glaucoma which target features of the disease other than or in addition to IOP.

### SUMMARY

Neuroserpin is a serine protease inhibitor which plays a role in inhibiting plasmin activity in the retina. The inventors have shown previously that oxidative inactivation of neuroserpin results in increased plasmin activity, and that decreased neuroserpin activity is associated with RGC degeneration and optic nerve damage, both of which are associated with glaucoma.

The inventors have now found that mutating methionine at position 363 of neuroserpin results in a mutant neuroserpin that retains the ability to inhibit plasmin activity and is resistant to oxidative inactivation. There is therefore provided a mutant neuroserpin that is at least partially resistant to inactivation by oxidative reduction. A first aspect provides a nucleic acid comprising a nucleotide sequence encoding a mutant neuroserpin protein or a portion thereof, the mutant neuroserpin or portion thereof comprising an amino acid sequence that is at least 60% identical to the amino acid sequence of SEQ ID NO: 2 and which comprises a reactive centre loop comprising the amino acid sequence MIAX₃SRX₁X₂VL (SEQ ID NO: 14), wherein:

X₁ is selected from R, S, T, F, N, E, K and D;

X₂ is M or A; and

X₃ is I, V or N,

and wherein the mutant neuroserpin protein or portion thereof has serine protease inhibition activity and is at least partially resistant to oxidative inactivation.

A second aspect provides a nucleic acid comprising a nucleotide sequence encoding a mutant neuroserpin protein or a portion thereof, the mutant neuroserpin or portion thereof comprising a reactive centre loop comprising the amino acid sequence MIAISRX₁AVL (SEQ ID NO: 15), wherein X₁ is selected from R, S, T, F, N, E, K and D.

A third aspect provides a nucleic acid comprising a nucleotide sequence encoding a mutant neuroserpin protein or a portion thereof, the mutant neuroserpin or portion thereof comprising a reactive centre loop comprising the amino acid sequence MIAISRRAVL (SEQ ID NO: 16).

A fourth aspect provides a nucleic acid comprising a nucleotide sequence encoding a mutant neuroserpin protein comprising an amino acid substitution of methionine at position 363 of wild-type neuroserpin.

A fifth aspect provides a nucleic acid comprising a nucleotide sequence encoding a mutant neuroserpin protein or a portion thereof, wherein the mutant neuroserpin comprises the amino acid sequence of SEQ ID NO: 2.

A sixth aspect provides a nucleic acid comprising a nucleotide sequence encoding a mutant neuroserpin protein or a portion thereof, the mutant neuroserpin or portion thereof comprising an amino acid sequence which differs from the corresponding wild-type neuroserpin in at least a substitution of methionine in the amino acid sequence IAISRMAVL (SEQ ID NO: 17) of the wild-type neuroserpin, wherein the mutant neuroserpin protein or portion thereof has serine protease inhibition activity and is at least partially resistant to oxidative inactivation.

A seventh aspect provides a nucleic acid comprising a nucleotide sequence encoding a mutant neuroserpin protein or a portion thereof, the mutant neuroserpin or portion thereof comprising an amino acid sequence which differs from the corresponding wild-type neuroserpin in at least a substitution of methionine in the amino acid sequence IAISRMAVLYPQV (SEQ ID NO: 18) of the wild-type neuroserpin, wherein the mutant neuroserpin protein or portion thereof has serine protease inhibition activity and is at least partially resistant to oxidative inactivation.

An eighth aspect provides a nucleic acid comprising a nucleotide sequence encoding a mutant neuroserpin protein or a portion thereof, the mutant neuroserpin or portion thereof comprising an amino acid sequence that is at least 60% identical to the amino acid sequence of SEQ ID NO: 2, and comprises the amino acid sequence MIAX₃SRX₁X₂VL (SEQ ID NO: 34), wherein:

X₁ is an amino acid other than M;

X₂ is M or A; and

X₃ is I, V or N,

and wherein the mutant neuroserpin protein or portion thereof has serine protease inhibition activity and is at least partially resistant to oxidative inactivation.

A ninth aspect provides a vector comprising the nucleic acid of any one of the first to eighth aspects.

A tenth aspect provides a viral particle comprising the vector of the ninth aspect.

An eleventh aspect provides a mutant neuroserpin protein or a portion thereof, comprising an amino acid sequence that is at least 60% identical to the amino acid sequence of SEQ ID NO: 2 and comprising a reactive centre loop comprising the amino acid sequence MIAX₃SRX₁X₂VL (SEQ ID NO: 14), wherein:

X₁ is selected from R, S, T, F, N, E, K and D;

X₂ is M or A; and

X₃ is I, V or N,

and wherein the mutant neuroserpin protein or portion thereof has serine protease inhibition activity and is at least partially resistant to oxidative inactivation.

A twelfth aspect provides a mutant neuroserpin protein or a portion thereof, the mutant neuroserpin or portion thereof comprising a reactive centre loop comprising the amino acid sequence MIAISRX₁AVL (SEQ ID NO: 15), wherein X₁ is selected from R, S, T, F, N, E, K and D.

A thirteenth aspect provides a mutant neuroserpin protein or a portion thereof, the mutant neuroserpin or portion thereof comprising a reactive centre loop comprising the amino acid sequence MIAISRRAVL (SEQ ID NO: 16).

A fourteenth aspect provides a mutant neuroserpin protein comprising an amino acid substitution of methionine at position 363 of wild-type neuroserpin.

A fifteenth aspect provides a mutant neuroserpin protein comprising the amino acid sequence of SEQ ID NO: 2.

A sixteenth aspect provides a mutant neuroserpin protein or a portion thereof, the mutant neuroserpin or portion thereof comprising an amino acid sequence which differs from the corresponding wild-type neuroserpin in at least a substitution of methionine in the amino acid sequence IAISRMAVL (SEQ ID NO: 17) of the wild-type neuroserpin, wherein the mutant neuroserpin protein or portion thereof has serine protease inhibition activity and is at least partially resistant to oxidative inactivation.

An seventeenth aspect provides a mutant neuroserpin protein or a portion thereof, the mutant neuroserpin or portion thereof comprising an amino acid sequence which differs from the corresponding wild-type neuroserpin in at least a substitution of methionine in the amino acid sequence IAISRMAVLYPQV (SEQ ID NO: 18) of the wild-type neuroserpin, wherein the mutant neuroserpin protein or portion thereof has serine protease inhibition activity and is at least partially resistant to oxidative inactivation.

An eighteenth aspect provides a mutant neuroserpin protein or a portion thereof, the mutant neuroserpin or portion thereof comprising an amino acid sequence that is at least 60% identical to the amino acid sequence of SEQ ID NO: 2 and comprises the amino acid sequence MIAX₃SRX₁X₂VL (SEQ ID NO: 34), wherein:

X₁ is an amino acid other than M;

X₂ is M or A; and X₃ is I, V or N,

and wherein the mutant neuroserpin protein or portion thereof has serine protease inhibition activity and is at least partially resistant to oxidative inactivation.

A nineteenth aspect provides a pharmaceutical composition comprising the nucleic acid of any one of the first to eighth aspects, a vector of the ninth aspect, a viral particle of the tenth aspect, or a mutant neuroserpin protein or portion thereof of any one of the eleventh to eighteenth aspects.

A twentieth aspect provides a method of treating or preventing a condition associated with elevated plasmin, tPA or uPA activity in a subject, comprising administering an effective amount of the nucleic acid of any one of the first to eighth aspects, a vector of the ninth aspect, a viral particle of the tenth aspect, or a mutant neuroserpin protein or portion thereof of any one of the eleventh to eighteenth aspects, or a composition of the nineteenth aspect.

An alternative twentieth aspect provides the nucleic acid of any one of the first to eighth aspects, a vector of the ninth aspect, a viral particle of the tenth aspect, or a mutant neuroserpin protein or portion thereof of any one of the eleventh to eighteenth aspects, or a composition of the ninteenth aspect, for use in treating or preventing a condition associated with elevated plasmin, tPA or uPA activity in a subject; or use of the nucleic acid of any one of the first to eighth aspects, a vector of the ninth aspect, a viral particle of the tenth aspect, or a mutant neuroserpin protein or portion thereof of any one of the eleventh to eighteenth aspects, or a composition of the ninteenth aspect, in the manufacture of a medicament for treating or preventing a condition associated with elevated plasmin, tPA or uPA activity in a subject.

A twenty first aspect provides a method of treating or preventing glaucoma in a subject, comprising administering an effective amount of the nucleic acid of any one of the first to eighth aspects, a vector of the ninth aspect, a viral particle of the tenth aspect, or a mutant neuroserpin protein or portion thereof of any one of the eleventh to eighteenth aspects, or a composition of the ninteenth aspect.

An alternative twenty first aspect provides the nucleic acid of any one of the first to eighth aspects, a vector of the ninth aspect, a viral particle of the tenth aspect, or a mutant neuroserpin protein or portion thereof of any one of the eleventh to eighteenth aspects, or a composition of the ninteenth aspect, for use in treating or preventing glaucoma in a subject; or use of the nucleic acid of any one of the first to eighth aspects, a vector of the ninth aspect, a viral particle of the tenth aspect, or a mutant neuroserpin protein or portion thereof of any one of the eleventh to eighteenth aspects, or a composition of the ninteenth aspect, in the manufacture of a medicament for treating or preventing glaucoma in a subject.

A twenty second aspect provides a kit comprising the nucleic acid of any one of the first to eighth aspects, a vector of the ninth aspect, a viral particle of the tenth aspect, or a mutant neuroserpin protein or portion thereof of any one of the eleventh to eighteenth aspects, or a composition of the ninteenth aspect.

### BRIEF DESCRIPTION OF THE FIGURES

FIG. 1 shows A. the amino acid sequence of full length wild-type human neuroserpin (UniProtKB-Q99574 (NS_Human) (SEQ ID NO: 1); and an example of a mutant neuroserpin (SEQ ID NO: 2). The mutation at Arg 363 residue is indicated as B. a schematic map of pSF-CAG plasmid carrying EGFP; C. a map of pSF-CAG plasmid carrying WT neuroserpin; D. A map of pSF-CAG plasmid carrying mutant neuroserpin,

FIG. 2 is an image of a western blot showing expression of wild-type (WT NS) or mutant (Mut NS) neuroserpin following expression in retinoic acid differentiated SH-SY5Y cell lysates. Actin is shown as loading control. The molecular weight of the proteins is shown in kDa.

FIG. 3 is an image of western blots showing immunoreactivity with an anti-methionine sulfoxide antibody for wild type and mutant neuroserpin proteins following treatment with hydrogen peroxide in vitro. The retained plasmin inhibitory activity (PIA) in mutant neuroserpin following H₂O₂ treatment is shown using gelatin gel zymography.

FIG. 4 is graphs showing A. Quantification of the plasmin inhibitory activity (PIA) of wild-type and mutant neuroserpin with and without H₂O₂ treatment following gelatin gel zymography; and B. Quantification of the methionine sulfoxide immunoreactivity for wild-type and mutant neuroserpin proteins with and without H₂O₂ treatment as shown in FIG. 3.

FIG. 5 is an image of Western blots showing neuroserpin protein expression is SHSY5Y cell lysates is shown following transfection with either WT and mutant neuroserpin plasmids, with or without the H₂O₂ treatment. The plasmin inhibitory activity (PIA) in the lysates is also shown under native non-denaturing conditions using gelatin gel zymography. Actin was used a loading control. The molecular weight of neuroserpin and actin is shown in kDa.

FIG. 6 is graphs showing quantification of plasmin inhibitory activity (PIA) of WT and mutant neuroserpin following H₂O₂ treatment of transfected SHSY5Y cells and subjecting the lysates to gelatin gel zymography. The PIA changes were plotted with respect to either A. total neuroserpin expression or B. actin expression in lysates as shown in FIG. 5. FIG. 6C is a graph showing quantification of western blots of neuroserpin expression in cell lysates in WT and mutant neuroserpin expressing groups from FIG. 5 with respect to actin.

FIG. 7 is an image of a western blot analysis of the SHSY5Y cell lysates for neuroserpin and methionine sulfoxide reactivity (Met S) following transfection with either WT or neuroserpin plasmids. The cells were either control group or treated with H₂O₂ to induce oxidative stress. Actin was used as loading control in each case.

FIG. 8 is a graph showing quantification of the methionine sulfoxide (Met S) western blot band intensities in FIG. 7. The Met S reactivity is plotted as a ratio to the total neuroserpin expression following various treatments.

FIG. 9 is a graph showing intraocular pressure (TOP) measurements following weekly microbead injection for 8 weeks in mice groups receiving either WT or mutant neuroserpin protein. The control group IOPs receiving sham injections is also shown. The IOPs were measured using rebound iCare Tonometer (TonoLab).

FIG. 10A. shows retinal electrophysiology traces indicating effect of treatment with either wild-type or mutant neuroserpin as measured using positive scotopic threshold response (pSTR) amplitudes in a mouse model of glaucoma.

FIG. 10B is a graph showing the quantification of the effect of wild-type and mutant neuroserpin treatment on pSTR amplitudes in a mouse model of glaucoma.

FIG. 11 is images of a cross-section of the retina stained with hematoxylin and eosin (H and E) of healthy mice and mice suffering from experimental glaucoma, with no treatment, and following treatment with wild-type or mutant neuroserpin proteins.

FIG. 12 is a graph showing the quantification of the number of cells in the ganglion cell layer (GCL) in a mouse model of glaucoma (as shown in FIG. 12), following no treatment, treatment with wild-type neuroserpin, or treatment with mutant neuroserpin protein.

FIG. 13 is western blot images showing levels of methionine sulfoxide (MetS), and plasmin inhibitory activity (PIA) in mice retinas suffering from glaucoma following no treatment, treatment with wild-type neuroserpin or treatment with mutant neuroserpin. Actin was used as loading control. The molecular weight of proteins is shown as kDa.

FIG. 14 is a graph showing the percent density change of plasmin inhibitory activity (PIA) as a ratio to total neuroserpin following gelatin gel zymography in a mouse model of glaucoma following no treatment, and treatment with either wild-type or mutant neuroserpin.

FIG. 15A shows retinal positive scotopic threshold response (pSTR) traces of WT and neuroserpin knockout mice. FIG. 15B shows pSTR electrophysiology traces of the WT and neuroserpin knockout mice that are subjected to experimental glaucoma following microbead injections. The pSTR traces of glaucoma neuroserpin knockout mice subjected to either WT or mutant neuroserpin protein administration are also shown.

FIG. 16 is a graph showing quantification of the pSTR amplitudes of various WT and neuroserpin knockout mice groups (as shown in FIG. 15) subjected to either WT or mutant neuroserpin administration.

FIG. 17 is images showing retinal sections subjected to hemtoxylin and eosin (H and E) staining in WT and neuroserpin knockout mice groups. The retinal sections of neuroserpin knockout mice subjected to experimental glaucoma paradigm with either WT neuroserpin or mutant neuroserpin protein administration are also shown. The white arrows indicate changes in the ganglion cell layer (GCL).

FIG. 18 is a graph showing quantification of cells in the ganglion cell layer (GCL) in the retinal sections of WT and neuroserpin knockout mice (as shown in FIG. 17). Changes in the GCL of neuroserpin knockout mice that were subjected to increased intraocular pressure (TOP) and administered either WT or neuroserpin protein are also shown. The GCL changes were quantified at distance of 0-500 μM from the optic nerve head and plotted as percent change.

FIG. 19 is a schematic diagram showing predicted hydrogen bond interactions between wild-type neuroserpin and tPA or M363R mutant neuroserpin and tPA.

FIG. 20 is a schematic diagram showing predicted salt bridge formation between wild-type neuroserpin and tPA or M363R mutant neuroserpin and tPA.

FIGS. 21A and C show retinal positive scotopic threshold response (pSTR) electrophysiology traces following treatment of mice with AAV mediated gene therapy to overexpress wild-type or mutant neuroserpin in RGCs of mice with (C) and without (A) experimental glaucoma induced degeneration. Overexpression of mutant human neuroserpin (M363R) imparted significant retinal function protection against experimental glaucoma induced degeneration. FIGS. 21B and D are graphs showing quantification of the pSTR amplitudes of following treatment of mice with AAV mediated gene therapy to overexpress wild-type or mutant human neuroserpin in RGCs of mice with (B) and without (A) experimental glaucoma induced degeneration (as shown in FIG. 21A and C).

FIGS. 22A and C show retinal sections subjected to hematoxylin and eosin staining from mice with (C) and without (A) experimental glaucoma induced degeneration and treated with AAV mediated gene therapy to overexpress wild-type or mutant human neuroserpin in RGCs of the mice. The white arrows indicate changes in the ganglion cell layer (GCL).

FIG. 22B and D shows quantification of the number of cells in the ganglion cell layer (GCL) in the retinal sections of mice (as shown in FIGS. 22A and C).

FIG. 23 is an alignment of wild-type neuroserpin from various species as indicated. The methionine at position 363 is underlined in each species.

FIG. 24 is the nucleotide sequence of cDNA clone MGC:26301 encoding homo sapiens wild-type neuroserpin, clade 1 (IMAGE:4796470; Accession no. BC018043). The coding sequence is underlined.

### DETAILED DESCRIPTION

Neuroserpin is a serine protease inhibitor which inhibits the activity of the serine proteases tissue plasminogen activator (tPA), plasmin, and urokinase-type plasminogen activator (uPA).

The inventors have previously shown that oxidative inactivation of neuroserpin is associated with increased plasmin activity, retinal ganglion cell (RGC) degeneration and excavation of the optic nerve head in glaucoma.

The inventors have reasoned that a neuroserpin that is resistant to oxidative inactivation may be advantageous for reducing plasmin activity, and that such a molecule could be used for reducing RGC degeneration and/or reducing excavation of the optic nerve head. The inventors therefore envisage that such a molecule could be used to treat or prevent glaucoma, and other conditions associated with plasmin activity or plasmin activator activity.

The inventors have found that oxidative inactivation of neuroserpin can be reduced or prevented by mutating a methionine residue at position 363 of wild-type neuroserpin. As described in the Examples, the inventors have found that substituting methionine at position 363 for arginine results in a mutant neuroserpin protein that is resistant to inactivation by oxidative inactivation, and retains the ability to inhibit serine proteases such as plasmin, tissue plasminogen activator, and urokinase plasminogen activator. The methionine at position 363 resides in the reactive centre loop of neuroserpin. The amino acid sequence of mutant human neuroserpin with a M363R is shown in SEQ ID NO: 2.

The reactive centre loop of neuroserpin plays a key role in the interaction of neuroserpin with serine proteases and their inactivation. In this regard, the reactive centre loop is believed to act as a bait for the target serine protease, wherein the target serine protease binds and cleaves the reactive centre loop at the bond P1-P1′ between amino acid residues R362 and M363, and forms an acyl-enzyme complex with the reactive centre loop. The cleavage causes a conformational change, triggering insertion of the reactive centre loop as a new strand of the main central β-sheet of the neuroserpin molecule, leading to translocation and inactivation of the protease.

In mammalian species, the reactive centre loop of wild-type neuroserpin comprises the amino acid sequence IAISRMAVL at position 358 to 366 which is highly conserved between a broad range of mammalian species, including for example, human, mouse, rat, chimpanzee, pig, cattle and Rhesus monkey, as well as some non-mammalian species (e.g., chicken). Among broader species, the sequence IAX₃SRMX₂VL (wherein X₂ is M or A; and X₃ is I, V or N) (SEQ ID NO: 36) at position 358 to 366 is highly conserved across mammalian and other species including, for example, chicken and frog (Xenopus laevis) species. In view of the conserved nature of this sequence, and its central role in recognition and inhibition of serine proteases, it was not expected that substitution of the methionine at position 363 would result in a functional neuroserpin, let alone that such a substitution would protect the neuroserpin from oxidative inactivation.

In view of the inventors unexpected result with substitution of methionine (M) with arginine (R) at position 363 of neuroserpin, the inventors further investigated other amino acid substitutions in this position using in silico analysis and found that substitution of the methionine at position 363 with leucine (L), serine (S), threonine (T), asparagine (N), glutamic acid (E), or aspartic acid (D) is also predicted to result in a stable mutant neuroserpin. The inventors also believe that lysine (K) would also be a suitable substitute.

Accordingly, one aspect provides a mutant neuroserpin protein or a portion thereof, the mutant neuroserpin or portion thereof comprising a reactive centre loop comprising the amino acid sequence MIAISRX₁AVL, wherein X₁ is selected from R, S, T, F, N, E, K and D (SEQ ID NO: 15).

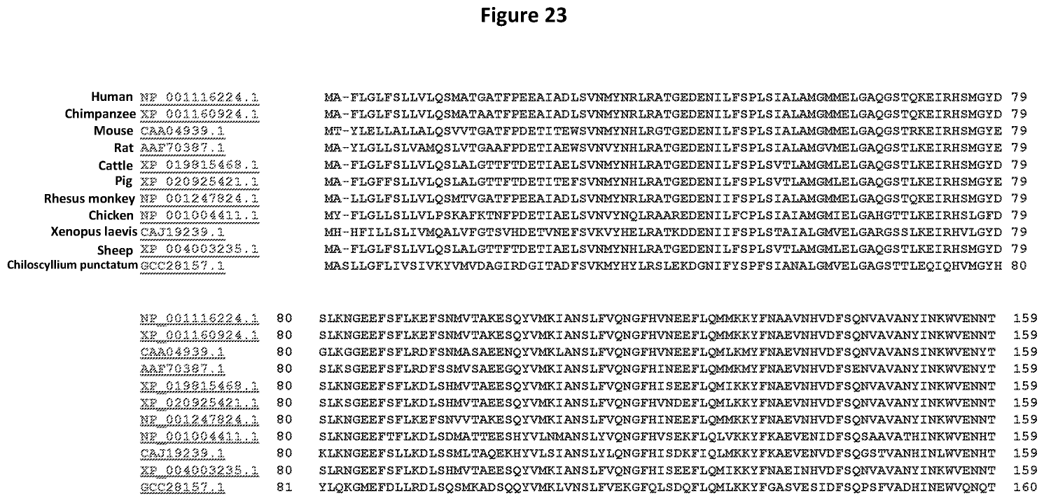

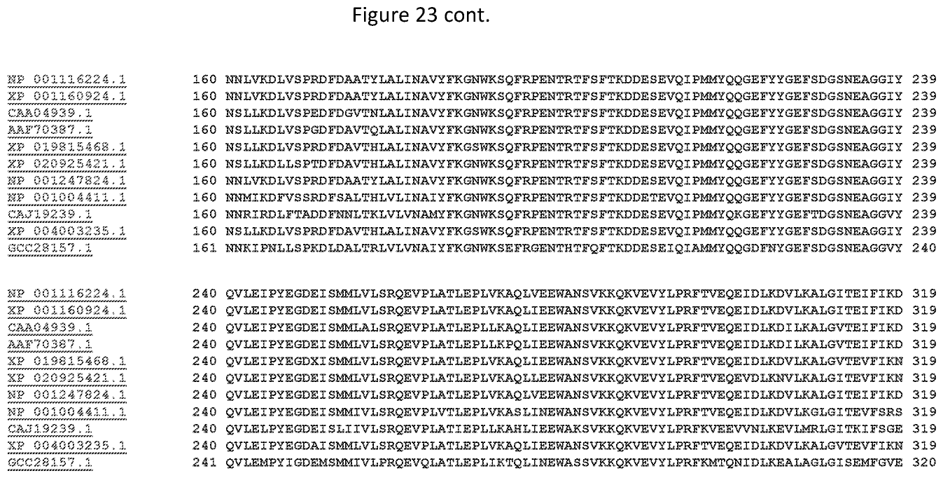

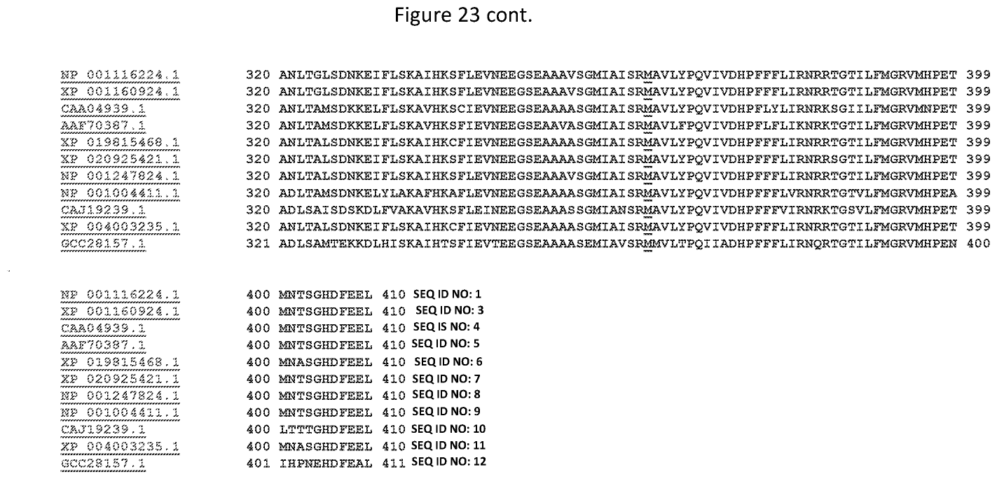

As shown in FIG. 23, the amino acid sequence from position 358 to position 366 of wild-type neuroserpin from various species is highly conserved and can be represented as IAX₃SRMX₂VL (wherein X₂ is M or A; and X₃ is I, V or N). The sequence IAISRMAVL (SEQ ID NO: 17) in the reactive centre loop of neuroserpin is highly conserved between neuroserpins from different mammalian species.

Table 1 sets out examples of the percent amino acid identity between full length neuropserpin from human and other species.

TABLE 1

| Species comparison | % identity* |
|---|---|
| Homo sapiens and Mus musculus | 86% |
| Homo sapiens and Rattus norvegicus | 88% |
| Homo sapiens and Gallus gallus | 80% |
| Homo sapiens and Xenopus laevis | 70% |
| Homo sapiens and Ovis aries | 91% |
| Homo sapiens and Pan troglodytes | 99% |
| Homo sapiens and Bos indicus | 91% |
| Homo sapiens and Sus scrofa | 90% |
| Homo sapiens and Macaca mulatta | 98% |
| Homo sapiens and Chiloscyllium | 61% |
| punctatum | |

*% identity determined using BLAST Global align base on Needleman-Wunsch algorithm (Needleman & Wunsch, (1970). Journal of Molecular Biology. 48 (3): 443-53).

One aspect provides a mutant neuroserpin protein or a portion thereof, comprising an amino acid sequence that is at least 60% identical to the amino acid sequence of SEQ ID NO: 2 and comprising a reactive centre loop comprising the amino acid sequence MIAX₃SRX₁X₂VL (SEQ ID NO: 14), wherein:

X₁ is an amino acid other than M; typically X₁ is selected from R, S, T, F, N, E, K, and D, more typically, X₁ is selected from R, S, T, F, N, E, and D;

X₂ is M or A; and

X₃ is I, V or N,

wherein the mutant neuroserpin protein or portion thereof has serine protease inhibition activity and is at least partially resistant to oxidative inactivation.

One aspect provides a mutant neuroserpin protein or a portion thereof, the mutant neuroserpin or portion thereof comprising a reactive centre loop comprising the amino acid sequence MIAISRX₁AVL (SEQ ID NO: 15), wherein X₁ is an amino acid other than M. Typically X₁ is selected from R, S, T, F, N, E, K, and D. More typically, X₁ is selected from R, S, T, F, N, E, and D.

Another aspect provides a mutant neuroserpin protein comprising an amino acid substitution of methionine at position 363 of wild-type neuroserpin with arginine, leucine, serine, threonine, asparagine, glutamic acid, lysine or aspartic acid.

Another aspect provides a mutant neuroserpin protein or a portion thereof, the mutant neuroserpin or portion thereof comprising an amino acid sequence which differs from the corresponding wild-type neuroserpin in a substitution of methionine in the amino acid sequence IAX₃SRMAVL (SEQ ID NO: 19), wherein X₃ is I, V or N, more typically the amino acid sequence IAISRMAVL (SEQ ID NO: 17), still more typically the amino acid sequence IAISRMAVLYPQV (SEQ ID NO: 18), of the wild-type neuroserpin, wherein the mutant neuroserpin protein or portion thereof has serine protease inhibition activity and is at least partially resistant to oxidative inactivation.

In one embodiment, the mutant neuroserpin protein or a portion thereof comprises the amino acid sequence MIAX₃SRX₁AVL, wherein: X₁ is an amino acid other than M, typically an amino acid selected from R, S, T, F, N, E, K, and D, more typically an amino acid selected from R, S, T, F, N, E, and D, still more typically R; and X₃ is I, V or N, more typically I.

In one embodiment, the mutant neuroserpin protein or a portion thereof comprises an amino acid sequence which differs from the corresponding wild-type neuroserpin in a substitution of methionine in the amino acid sequence IAX₃SRMAVL (SEQ ID NO: 19), wherein X₃ is I, V or N, more typically the amino acid sequence IAISRMAVL (SEQ ID NO: 17), still more typically the amino acid sequence IAISRMAVLYPQV (SEQ ID NO: 18), such that the mutant neuroserpin or portion thereof comprises the amino acid sequence IAX₃SRX₁AVL (SEQ ID NO: 37), wherein: X₁ is an amino acid other than M, typically an amino acid selected from R, S, T, F, N, E, K, and D, more typically and amino acid selected from R, S, T, F, N, E, and D, still more typically R; and X₃ is I, V or N, more typically I,

wherein the mutant neuroserpin protein or portion thereof has serine protease inhibition activity, and is at least partially resistant to oxidative inactivation.

Typically, the serine protease inhibition activity is inhibition of plasmin, tissue plasminogen activator and/or urokinase plasminogen activator.

Another aspect provides a nucleic acid comprising a nucleotide sequence encoding a mutant neuroserpin protein or a portion thereof.

Accordingly, another aspect provides a nucleic acid comprising a nucleotide sequence encoding a mutant neuroserpin protein or a portion thereof, the mutant neuroserpin protein or a portion thereof comprising an amino acid sequence that is at least 60% identical to the amino acid sequence of SEQ ID NO: 2 and comprising a reactive centre loop comprising the amino acid sequence MIAX₃SRX₁X₂VL (SEQ ID NO: 14), wherein:

X₁ is an amino acid other than M, typically an amino acid selected from R, S, T, F, N, E, K, and D, more typically an amino acid selected from R, S, T, F, N, E, and D;

X₂ is M or A; and

X₃ is I, V or N,

and wherein the mutant neuroserpin protein or portion thereof has serine protease inhibition activity and is at least partially resistant to oxidative inactivation.

Another aspect provides a nucleic acid comprising a nucleotide sequence encoding a mutant neuroserpin protein or a portion thereof, the mutant neuroserpin or portion thereof comprising an amino acid sequence which differs from the corresponding wild-type neuroserpin in a substitution of methionine in the amino acid sequence IAX₃SRMAVL (SEQ ID NO: 19), wherein X₃ is I, V or N, more typically the amino acid sequence IAISRMAVL (SEQ ID NO: 17), still more typically the amino acid sequence IAISRMAVLYPQV (SEQ ID NO: 18), of the wild-type neuroserpin, wherein the mutant neuroserpin protein or portion thereof has serine protease inhibition activity and is at least partially resistant to oxidative inactivation.

In one embodiment, the mutant neuroserpin protein or a portion thereof comprises the amino acid sequence MIAX₃SRX₁AVL, wherein: X₁ is an amino acid other than M, typically an amino acid selected from R, S, T, F, N, E, K, and D, more typically an amino acid selected from R, S, T, F, N, E, and D, still more typically R; and X₃ is I, V or N, more typically I.

One aspect provides a nucleic acid comprising a nucleotide sequence encoding a One aspect provides a mutant neuroserpin protein or a portion thereof, the mutant neuroserpin or portion thereof comprising an amino acid sequence which differs from the corresponding wild-type neuroserpin in a substitution of methionine in the amino acid sequence IAX₃SRMAVL (SEQ ID NO: 19), wherein X₃ is I, V or N, more typically the amino acid sequence IAISRMAVL (SEQ ID NO: 17), still more typically the amino acid sequence IAISRMAVLYPQV (SEQ ID NO: 18), such that the mutant neuroserpin or portion thereof comprises the amino acid sequence IAX₃SRX₁AVL, wherein: X₁ is an amino acid other than M, typically an amino acid selected from R, S, T, F, N, E, K, and D, more typically an amino acid selected from R, S, T, F, N, E, and D, still more typically R; and X₃ is I, V or N, more typically I,

wherein the mutant neuroserpin protein or portion thereof has serine protease inhibition activity, and is at least partially resistant to oxidative inactivation.

In various embodiments, the amino acid sequence of the mutant neuroserpin protein is at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% identical to the amino acid sequence of SEQ ID NO: 2. In one embodiment, the amino acid sequence of the mutant neuroserpin protein is identical to the amino acid sequence of SEQ ID NO: 1 except for a single amino acid at position 363. In one embodiment, the amino acid sequence of the mutant neuroserpin protein is identical to the amino acid sequence of SEQ ID NO: 1 except for substitution of methionine at 363 to arginine. More typically, the amino acid sequence of the mutant neuroserpin protein is identical to the amino acid sequence of SEQ ID NO: 1 except for substitution of methionine at 363 to arginine.

In various embodiments, le mutant neuroserpin or portion thereof comprises the amino acid sequence:
(a) MIAX₃SRX₁X₂VL (SEQ ID NO: 14), wherein: X₁ is selected from R, S, T, F, N, E, K and D, typically selected from R, S, T, F, N, E, and D, more typically R; X₂ is selected from A and M, typically A; and X₃ is selected from I, V and N, typically I;
(b) MIAX₃SRX₁AVLX₄(SEQ ID NO: 20), wherein: X₁ is selected from R, S, T, F, N, E, K and D, typically selected from R, S, T, F, N, E, and D, more typically R; X₃ is selected from I, V and N, typically I; and X₄ is Y or F;
(c) MIAX₃SRX₁AVLX₄PQV (SEQ ID NO: 21), wherein: X₁ is selected from R, S, T, F, N, E, K and D, typically selected from R, S, T, F, N, E, and D, more typically R; X₃ is selected from I, V and N, typically I; and X₄ is Y or F;
(d) MIAISRX₁AVLX₄PQVI (SEQ ID NO: 22), wherein: X₁ is selected from R, S, T, F, N, E, K and D, typically selected from R, S, T, F, N, E, and D, more typically R; and X₄ is Y or F;
(e) GMIAISRX₁AVLX₄PQVIV (SEQ ID NO: 23), wherein: X₁ is selected from R, S, T, F, N, E, K and D, typically selected from R, S, T, F, N, E, and D, more typically R; and X₄ is Y or F;
(f) SGMIAISRX₁AVLX₄PQVIVDHPF (SEQ ID NO: 24), wherein: X₁ is selected from R, S, T, F, N, E, K and D, typically selected from R, S, T, F, N, E, and D, more typically R, and X₄ is Y or F;

(g)
(SEQ ID NO: 25)
IAISRRAVLYPQV;
(h)
(SEQ ID NO: 26)
MIAISRRAVLYPQVI;
(i)
(SEQ ID NO: 27)
GMIAISRRAVLYPQVIV;
(j)
(SEQ ID NO: 28)
SGMIAISRRAVLYPQVIVDHPF;
(k)
(SEQ ID NO: 29)
EAAAVSGMIAISRRAVLYPQVIVDHPFFFLIRNR;
RTG
(l)
(SEQ ID NO: 30)
LEVNEEGSEAAAVSGMIAISRRAVLYPQVIVDHP
FFFLIRNRRTGTILFMGR
(m)
(SEQ ID NO: 31)
SKAIHKSFLEVNEEGSEAAAVSGMIAISRRAVLYP
QVIVDHPFFFLIRNRRTGTILFMGRVMHPETM;
and/or
(n)
(SEQ ID NO: 32)
NLTGLSDNKEIFLSKAIHKSFLEVNEEGSEAAAVS
GMIAISRRAVLYPQVIVDHPFFFLIRNRRTGTILFM
GRVMHPETMNTSGHDFEEL.

In one embodiment, X₁ is R.

In one embodiment, X₁ is S.

In one embodiment, X₁ is T.

In one embodiment, X₁ is F.

In one embodiment, X₁ is N.

In one embodiment, X₁ is E.

In one embodiment, X₁ is D.

In one embodiment, X₁ is K.

In one embodiment, X₁ is R, X₂ is X₃ is I and X₄ is Y.

In one embodiment, X₁ is R, X₂ is A, X₃ is I and X₄ is F.

In one embodiment, X₁ is R, X₂ is A, X₃ is V and X₄ is Y.

In one embodiment, X₁ is R, X₂ is A, X₃ is V and X₄ is F.

In one embodiment, X₁ is R, X₂ is A, X₃ is N and X₄ is Y.

In one embodiment, X₁ is R, X₂ is A, X₃ is N and X₄ is F.

In one embodiment, the amino acid sequence of the mutant neuroserpin may correspond to the amino acid sequence of neuroserpin from human or non-human.

In one embodiment, the mutant neuroserpin comprises the amino acid sequence of human neuroserpin in which the methionine at position 363 is substituted.

In some embodiments, the mutant neuroserpin comprises the amino acid sequence of non-human neuroserpin in which the methionine at position 363 is substituted. Examples of non-human neuroserpin include mammalian or non-mammalian non-humans. Examples of mammalian non-humans include rat, mouse, cattle, pig, chimpanzee, Rhesus monkey. Examples of non-mammalian non-humans include chicken, and Xenopus laevis. Typically, the non-human neuroserpin is mouse or rat.

In some embodiments, the amino acid sequence of the mutant neuroserpin protein is identical to the amino acid sequence of SEQ ID NO: 3, 4, 5, 6, 7, 8, 9, 10, or 11 except for a single amino acid substitution of methionine at position 363. In some embodiments, the amino acid sequence of the mutant neuroserpin protein is identical to the amino acid sequence of SEQ ID NO: 3, 4, 5, 6, 7, 8, 9, 10, or 11 except for substitution of methionine at 363 to an amino acid selected from R, S, T, F, N, E, and D. In some embodiments, the amino acid sequence of the mutant neuroserpin protein is identical to the amino acid sequence of SEQ ID NO: 3, 4, 5, 6, 7, 8, 9, 10, or 11 except for substitution of methionine at 363 to arginine.

In one embodiment, the mutant neuroserpin protein comprises, consists essentially of, or consists of, the amino acid sequence:

(SEQ ID NO: 35)
MAFLGLFSLLVLQSMATGATFPEEAIADLSVNMYNRLRAT
GEDENILFSPLSIALAMGMMELGAQGSTQKEIRHSMGYDS
LKNGEEFSFLKEFSNMVTAKESQYVMKIANSLFVQNGFHV
NEEFLQMMKKYFNAAVNHVDFSQNVAVANYINKWVENNIN
NLVKDLVSPRDFDAATYLALINAVYFKGNWKSQFRPENTR
TF SFTKDDESEVQIPMMYQQGEFYYGEFSDGSNEAGGIY
QVLEIPYEGDEISMMLVLSRQEVPLATLEPLVKAQLVEEW
ANSVKKQKVEVYLPRFTVEQEIDLKDVLKALGITEIFIKD
ANLTGLSDNKEIFLSKAIHKSFLEVNEEGSEAAAVSGMIA
ISRX₁AVLYPQVIVDHPFFFLIRNRRTGTILFMGRVMHPE
TMNTSGHDFEEL

wherein X is an amino acid other than M, typically an amino acid selected from R, S, T, F, N, E, K, and D, more typically an amino acid selected from R, S, T, F, N, E, and D.

In one embodiment, X₁ is R.

In one embodiment, X₁ is S.

In one embodiment, X₁ is T.

In one embodiment, X₁ is F.

In one embodiment, X₁ is N.

Iii one embodiment, X₁ is E.

In one embodiment, X₁ is D.

In one embodiment, X₁ is K.

In one embodiment, the mutant neuroserpin protein comprises, consists essentially of, or consists of, the amino acid sequence of SEQ ID NO:2.

The methionine at position 363 of neuroserpin is a methionine corresponding to the methionine at position 363 of wild-type human neuroserpin (SEQ ID NO: 1). The methionine at position 363 is the methionine in the sequence IAX3SRMAVL (wherein X₃ is I, V or N) of neuroserpin.

In one embodiment, the nucleotide sequence encoding the mutant neuroserpin is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 87%, 83%, 84%, 85%, 86%, 87%, 88%, 89%, 90%, 91%, 92%, 93%, 94%, 95%, 96%, 97%, 98%, or at least 99% identical to the nucleotide sequence of SEQ ID NO: 13. In one embodiment, the nucleotide sequence encoding the mutant neuroserpin is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%. or at least 99% identical to the nucleotide sequence of SEQ ID NO: 13, and encodes the amino acid sequence represented by SEQ ID NO: 2. SEQ ID NO: 13 is the coding sequence of wild-type human neuroserpin and is shown below.

(SEQ ID NO: 13)
a tggctttcct tggactcttc
tctttgctgg ttctgcaaag tatggctaca
Ggggccactt tccctgagga agccattgct
gacttgtcag tgaatatgta taatcgtctt
agagccactg gtgaagatga aaatattctc
ttctctccat tgagtattgc tcttgcaatg
ggaatgatgg aacttggggc ccaaggatct
acccaggaag aaatccgcca ctcaatggga
tatgacagcc taaaaaatgg tgaagaattt
tctttcttga aggagttttc aaacatggta
actgctaaag agagccaata tgtgatgaaa
attgccaatt ccttgtttgt gcaaaatgga
tttcatgtca atgaggagtt tttgcaaatg
atgaaaaaat attttaatgc agcagtaaat
catgtggact tcagtcaaaa tgtagccgtg
gccaactaca tcaataagtg ggtggagaat
aacacaaaca atctggtgaa agatttggta
tccccaaggg attttgatgc tgccacttat
ctggccctca ttaatgctgt ctatttcaag
gggaactgga agtcgcagtt taggcctgaa
aatactagaa ccttttcttt cactaaagat
gatgaaagtg aagtccaaat tccaatgatg
tatcagcaag gagaatttta ttatggggaa
tttagtgatg gctccaatga agctggtggt
atctaccaag tcctagaaat accatatgaa
ggagatgaaa taagcatgat gctggtgctg
tccagacagg aagttcctct tgctactctg
gagccattag tcaaagcaca gctggttgaa
gaatgggcaa actctgtgaa gaagcaaaaa
gtagaagtat acctgcccag gttcacagtg
gaacaggaaa ttgatttaaa agatgttttg
aaggctcttg gaataactga aattttcatc
aaagatgcaa atttgacagg cctctatgat
aataaggaga tttttctttc caaagcaatt
cacaagtcct tcctagaggt taatgaagaa
ggctcagaag ctgctgctgt ctcaggaatg
attgcaatta gtaggatggc tgtgctgtat
cctcaagtta ttgtcgacca tccatttttc
tttcttatca gaaacaggag aactggtaca
attctattca tgggacgagt catgcatcct
gaaacaatga acacaagtgg acatgatttc
gaagaacttt aa

In some embodiments, the nucleic acid comprising nucleotide sequence encoding mutant neuroserpin protein comprises a regulatory sequence operatively linked to the nucleotide sequence encoding mutant neuroserpin for permitting expression of the mutant neuroserpin in a cell. The cell may be a prokaryotic or eukaryotic cell. In one embodiment, the cell is a prokaryotic cell. In one embodiment, the cell is a eukaryotic cell. Typically, the cell is a retinal ganglion cell (RGC). In some embodiments, the cells may be other cells in the retina such as photoreceptor cells (rods and cones), bipolar cells, amacrine cells, muller glial cells, horizontal cells and retinal pigment epithelial cells using appropriate promoters known in the art. The cells may be any other cells in the central nervous system comprising brain and the spinal cord, including for example, sensory neurons, motor neurons, interneurons, oligodendrocytes, microglia and astrocytes.

In one embodiment, the regulatory sequence comprises a promoter. A “promoter” refers to a nucleic acid (e.g., DNA) sequence that is located adjacent to a sequence, such as a nucleotide sequence that encodes a mutant neuroserpin protein or fragment thereof. A promoter that is operatively linked to a nucleic acid sequence typically increases the amount of mRNA expressed from that nucleic acid sequence compared to an amount expressed when no promoter exists under the same conditions. An “enhancer” can refer to a sequence that is located adjacent to the sequence, such as a nucleotide sequence that encodes a mutant neuroserpin protein or fragment thereof. Enhancer elements are typically located upstream of a promoter but also function and can be located downstream of a promoter, or be within a DNA sequence. Hence, an enhancer element can be located 10-25, 25-50, 50-100 100-200, 200-300 or more base pairs upstream or downstream of a sequence that encodes a mutant neuroserpin protein or fragment thereof. Enhancer elements typically also increase expression of an operatively linked sequence.

As used herein, the term “operable linkage” or “operably linked” refers to a physical or functional association of the components so that they function together in their intended manner. In the example of a promoter in operable linkage with a nucleic acid, the relationship is such that the promoter modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Any promoter may be employed that can express the mutant neuroserpin protein in the desired cell type. It will therefore be appreciated that the type of promoter employed will depend for what expression of the protein is intended. For example, in embodiments when expression is to produce mutant neuroserpin protein, the promoter may be a bacterial promoter, or a promoter for efficient expression in a suitable eukaryotic host cell such as a CHO cell. In embodiments when mutant neuroserpin is to be expressed in tissue, such as in gene therapy applications, the promoter may be of a type that is expressed in cells of the retina, such as in retinal ganglion cells, photoreceptor cells (rods and cones), bipolar cells, amacrine cells, muller glial cells, horizontal cells and retinal pigment epithelial cells. The promoter may be a cell specific, a constitutive or an inducible promoter, that can express the mutant neuroserpin protein-encoding or fragment-encoding nucleic acid in the desired cell type. In one embodiment, a promoter is cell-specific. The term “cell-specific” means that the particular promoter can direct expression of the operably linked coding sequence is a particular cell type. In one embodiment, the promoter is specific for RGCs. A promoter that is specific for RGCs can direct expression of the operably linked coding sequence (e.g., mutant neuroserpin) in RGCs, and typically does not express, or shows reduced expression of, the coding sequence in cell types that are unrelated to RGCs. Representative non-limiting examples of RGC-specific promoters include Brn3a, Nefh promoter, Thy1 promoter, GRM6 promoter, gamma-synuclein promoter, β-3 tubulin promoter.

Expression control elements include ubiquitous or promiscuous promoters/enhancers which are capable of driving expression in many different cell types, including RGCs. In one embodiment, the promoter is constitutive. Representative non-limiting examples of constitutive promoters include cytomegalovirus (CMV) immediate early enhancer/chicken β-actin (CβA) promoter-exon 1-intron 1 element, Rous sarcoma virus (RSV) promoter/enhancer sequences, LTR promoter/enhancer, SV40 promoter, CMV promoter, dihydrofolate reductase promoter, and phosphoglycerol kinase (PGK) promoter.

In some embodiments, the nucleic acid is DNA.

In some embodiments, the nucleic acid is RNA, typically mRNA.

In some embodiments, the nucleic acid is a combination of DNA and RNA.

The nucleic acid may be recombinant, produced using known methods such as, PCR, RT-PCR and/or cloning methods known in the art. In some embodiments, the nucleic acid may be synthetic. A synthetic nucleic acid is chemically synthesised. Methods for chemical synthesis of nucleic acid are known in the art.

The nucleic acid encoding the mutant neuroserpin or portion thereof may be codon optimized to optimised expression of the mutant neuroserpin in the host, using methods known in the art and described in, for example, U.S. Pat. No. 8,326,547; Papamichail, et al. (2018) Codon Context Optimization in Synthetic Gene Design. IEEE/ACM Transactions on Computational Biology and Bioinforrnatics 15: 452-459. Nuclei acid encoding a polypeptide that has been modified with respect to codon-usage, is optimized to obtain improved expression of the nucleotide sequence encoding the polypeptide and/or improved production of the encoded polypeptide.

The nucleic acid encoding the mutant neuroserpin or portion thereof may be modified. For example, in embodiments in which the nucleic acid is RNA, the RNA may be modified to reduce degradation and/or enhance translation of the RNA, and/or reduce innate immune sensing. Methods for modification of nucleic acid to reduce or prevent cellular degradation, enhance in vivo translation and reduce innate immune sensing are known in the art. Suitable modifications for mRNA include, for example, pseudouridine, N1-methylpseudouridine and/or 5-methoxyuridine modifications, 5′ untranslated region addition, 3′ untranslated region addition, poly A tail addition, and 5′ capping.

Nucleic acids encoding mutant neuroserpin protein may be incorporated into a vector.

Accordingly, another aspect provides a vector comprising a nucleic acid, wherein the nucleic acid comprises a nucleotide sequence encoding the mutant neuroserpin protein described herein. In such vectors, the nucleic acid comprising nucleotide sequence encoding the mutant neuroserpin protein is inserted into an appropriate vector sequence. The term “vector” refers to a nucleic acid sequence suitable for transferring genes into a host cell, such as a retinal ganglion cell. The term “vector” includes non-viral vectors, and viral vectors. In some embodiments, the vector is a non-viral vector. Examples of non-viral vectors include plasmids, cosmids, naked DNA, modified RNA. In one embodiment, the vector is a plasmid vector. A plasmid vector is a double stranded circular DNA molecule into which additional sequence may be inserted. The plasmid may be an expression vector. Plasmids vectors, including and expression vectors, are known in the art and described in, for example, Sambrook et al. Molecular Cloning: A Laboratory Manual, 4^{th} Ed. Vol. 1-3, Cold Spring Harbor, N.Y. (2012).

In some embodiments, the vector is a viral vector. Viral vectors comprise viral sequence which permits, depending on the viral vector, viral particle production and/or integration into the host cell genome and/or viral replication. Viral vectors which can be utilized with the methods and compositions described herein include any viral vector which is capable of introducing a nucleic acid into retinal cells, typically retinal ganglion cells, of the retina. Examples of viral vectors include adenovirus vectors; lentiviral vectors; adeno-associated viral vectors; Rabiesvirus vectors; Herpes Simplex viral vectors; SV40; polyoma viral vectors; poxvirus vector.

In one embodiment, the viral vector is an adeno-associated viral (AAV) vector. As described in the Examples, the inventors have shown that mutant M363R human neuroserpin can be effectively delivered to retinal cells in an AAV vector.

In one embodiment, the AAV vector is a serotype selected from AAV1, AAV2, AAV3, AAV4, AAV5, AAV6, AAV6.2, AAV7, AAV8, AAV9, AAVrh10, AAVrh20, AAVrh39, AAVrh43, and AAVcy5 vector or variants thereof. In one embodiment, the viral vector is serotype AAV1, AAV9, AAVrh10 or AAVcy5. In one embodiment, the serotype of the AAV vector is AAV1. In another embodiment, the serotype of the AAV vector is AAV9. In another embodiment, the serotype of the AAV vector is AAVrh10. In another embodiment, the serotype of the AAV vector is AAVcy5.

Typically, the viral vector comprises an AAV capsid sequence. Typically, the AAV capsid sequence comprises a VP1, VP2 and/or VP3 capsid sequence having at least 90% identity to the VP1, VP2 and/or VP3 sequences of AAV1, AAV2, AAV3, AAV4, AAV5, AAV6, AAV7, AAV8, AAV9, AAV10, AAV11, Rh10, Rh74 or AAV-2i8. Typically, the viral vector comprises one or more AAV inverted terminal repeat (ITR) sequences.

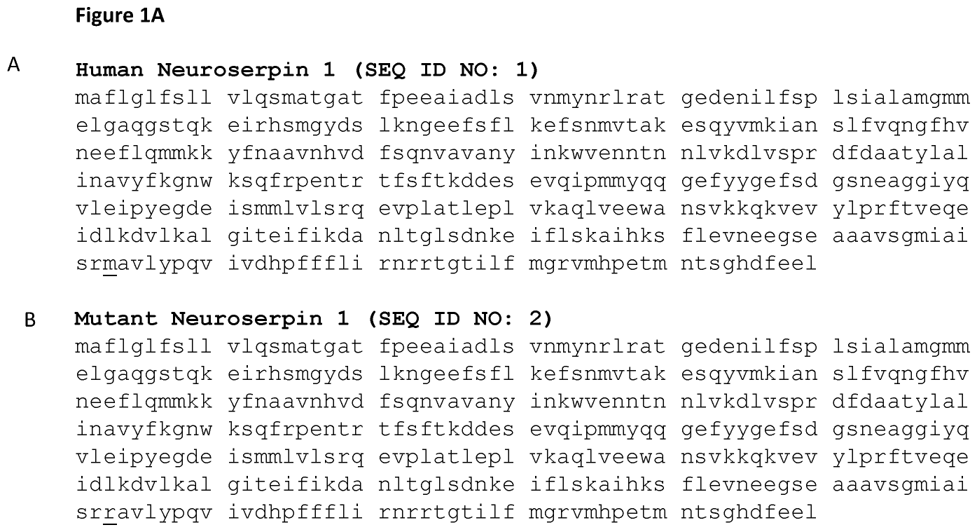

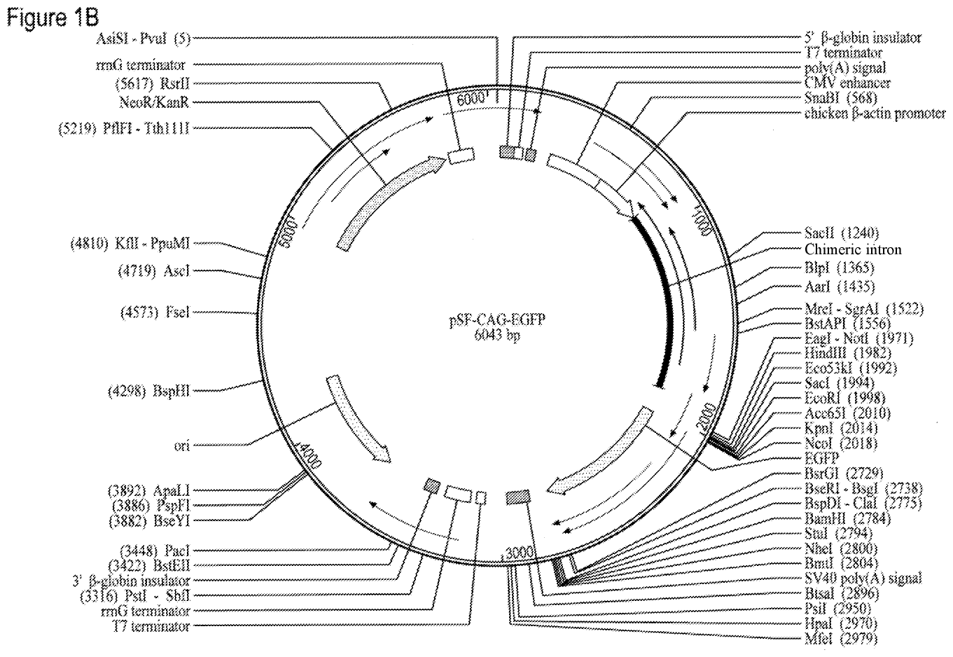

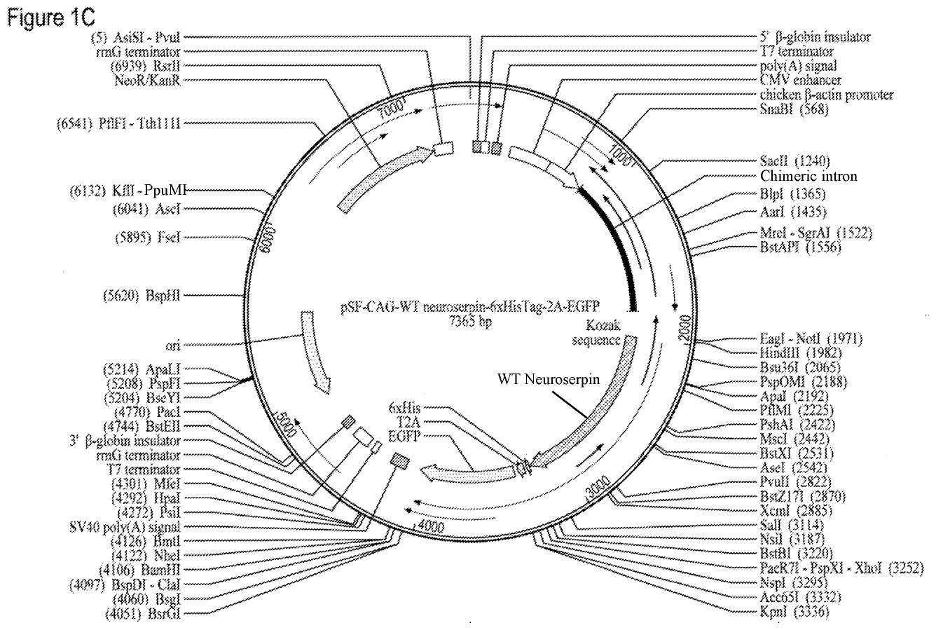

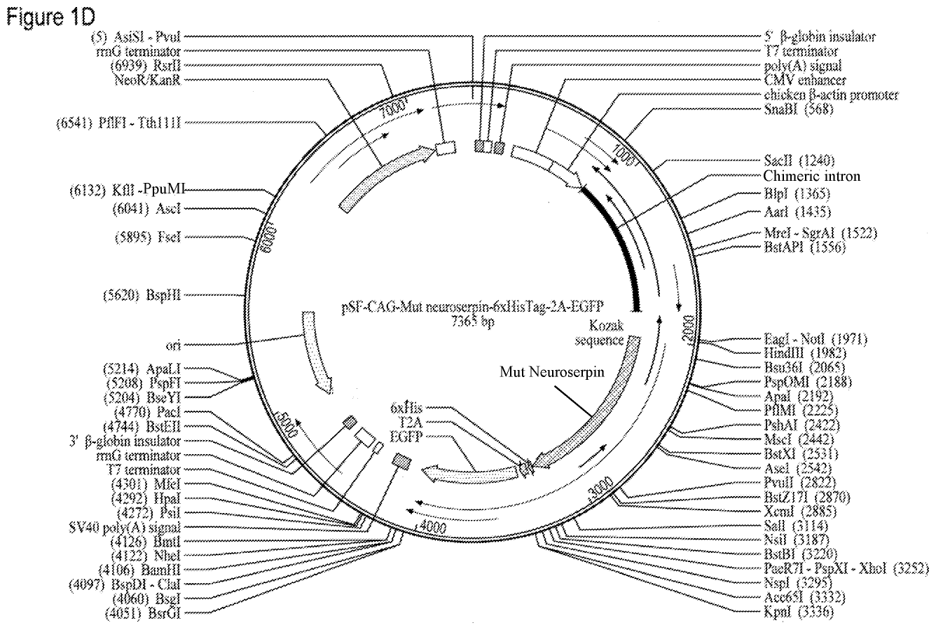

The use of recombinant AAV for introducing nucleic acids into cells is known in the art and described in, for example, US20160038613; Grieger and Samulski (2005) Adeno-associated virus as a gene therapy vector: vector development, production and clinical applications, Advances in Biochemical Engineering/Biotechnology 99: 119-145; Methods for the production of recombinant AAV are known in the art and described in, for example, Harasta et al (2015) Neuropsychopharmacology 40: 1969-1978. An example of an adeno-associated viral vector plasmid (pSF-CAG) capable of expressing mutant neuroserpin in RGCs is shown in FIG. 1D.

In another embodiment, the viral vector is a lentiviral vector. Methods for production and use of lentiviral vectors are known in the art and described in, for example, Naldini et al. (1996) In vivo gene delivery and stable transduction of nondividing cells by a lentiviral vector, Science, 272:263-267; Lois et al. (2002) Germline transmission and tissue-specific expression of transgenes delivered by lentiviral vectors, Science,295:868-872; Vogel et al (2004), A single lentivirus vector mediates doxycycline-regulated expression of transgenes in the brain. Hum Gene Ther. 2004;15(2):157-165.

Adenoviruses are also contemplated for use in delivery of nucleic acid agents. Thus, in another embodiment, the viral vector is an adenoviral vector. Adenoviral vectors are known in the art and described in, for example, Kozarsky and Wilson, Current Opinion in Genetics and Development 3:499-503 (1993); Southgate et al. (2008) Gene transfer into neural cells in vitro using adenoviral vectors, Current Protocols in Neuroscience, Unit 4 23, Chapter 4.

Vectors can include additional nucleic acid or protein elements. For example, an AAV vector can include one or two inverted terminal repeat (ITR) sequences of AAV genome retained in the AAV vector. ITR sequences can comprise or be based upon ITRs from any AAV serotype8. Typically, in an AAV vector the nucleic acid encoding mutant neuroserpin protein is flanked by 5′ and/or 3′ AAV ITR sequences. Additional non-limiting examples of nucleic acid sequences include introns, poly-adenine sequence, stop codon, etc. Such sequences including expression control elements can be located at the 5′ (i.e., “upstream”), 3′ end (i.e., “downstream”) of the transcribed sequence or within the sequence (e.g., in an intron).

Viral vectors are typically packaged into viral particles using methods known in the art. The viral particles may then be used for delivery of the mutant neuroserpin or nucleic acid encoding the mutant neuroserpin, into cells, including RGC, either in vitro or in vivo. Thus, another aspect provides a viral particle comprising a vector described herein.

The mutant neuroserpin protein, or nucleic acid encoding the mutant neuroserpin protein and vectors comprising the nucleic acid encoding the mutant neuroserpin protein described herein may be formulated for introduction into cells such as RGCs in vitro or in vivo by non-viral methods such as microinjection, electroporation, microparticle bombardment, nanoparticle-based delivery etc.

The mutant neuroserpin protein, or nucleic acid encoding the mutant neuroserpin protein and vectors comprising the nucleic acid encoding the mutant neuroserpin protein, may be packaged or prepared in other suitable forms for delivery into tissue and cells, such as in nanoparticles, liposomes, lipoplexes, exosomes, gold particles, or any other suitable delivery vehicles for administration to cells, typically cells of the eye.

In one embodiment, the mutant neuroserpin protein, or nucleic acid encoding the mutant neuroserpin protein and vectors comprising the nucleic acid encoding the mutant neuroserpin protein described herein are formulated in liposomes. Liposomes are unilamellar or multilamellar vesicles which have a membrane formed from a lipophilic material and an aqueous interior. The aqueous portion contains the composition to be delivered. Liposome design may include, for example, opsonins or ligands in order to improve the attachment of liposomes to tissue or to activate events such as, for example, endocytosis.

The formation of liposomes may depend on the physicochemical characteristics such as the agent and the liposomal ingredients, the nature of the medium in which the lipid vesicles are dispersed, the effective concentration of the agent, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

Methods for the production of liposomes and lipid nanoparticles for delivery of proteins and nucleic acids are known in the art, and described in, for example, (Wang Y et al. (2016) Cell-Specific Promoters Enable Lipid-Based Nanoparticles to Deliver Genes to Specific Cells of the Retina In Vivo. Theranostics, 6, 1514-27; Wang Yet al. (2015) Lipid Nanoparticles for Ocular Gene Delivery. J Funct Biomater, 6, 379-94; Lee, J. et al (2017). Effective Retinal Penetration of Lipophilic and Lipid-Conjugated Hydrophilic Agents Delivered by Engineered Liposomes. Mol Pharm, 14, 423-430; Adijanto, J et al. (2015). Nanoparticle-based technologies for retinal gene therapy. Eur J Pharm Biopharm, 95, 353-67; Urquhart, A. J. and Eriksen, A. Z. (2019). Recent developments in liposomal drug delivery systems for the treatment of retinal diseases. Drug Discov Today, 24, 1660-1668.

The mutant neuroserpin protein, nucleic acid encoding mutant neuroserpin, or vectors, viral particles or other delivery vehicles comprising the mutant neuroserpin protein or nucleic acid encoding mutant neuroserpin (collectively referred to herein as the agent described herein), may be formulated as a pharmaceutical composition. Accordingly, in another aspect, there is provided a pharmaceutical composition comprising the agent described herein. The composition comprises the agent in a pharmaceutically acceptable carrier. Methods for the formulation of agents with pharmaceutical carriers are known in the art and are described in, for example, Remington's Pharmaceutical Science, (17^{th} ed. Mack Publishing Company, Easton, Pa. 1985); Goodman & Gillman's: The Pharmacological Basis of Therapeutics (11^{th} Edition, McGraw-Hill Professional, 2005).

Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

As described in the Examples, the inventors have found that administration of mutant neuroserpin reduces plasmin activity in the eye of subjects suffering from glaucoma.

Accordingly, one aspect provides a method of treating or preventing glaucoma in a subject, comprising administering an effective amount of a mutant neuroserpin or a nucleic acid encoding a mutant neuroserpin protein described herein.

In various embodiments, the subject is administered:
(a) a mutant neuroserpin protein described herein; or
(b) a nucleic acid encoding a mutant neuroserpin protein described herein; or
(c) a vector comprising a nucleic acid encoding a mutant neuroserpin protein described herein; or
(d) a viral particle comprising a vector which comprises a nucleic acid encoding a mutant neuroserpin protein described herein;
(e) a liposome, exosome, nanoparticle, gold particle, or lipoplex, comprising any one of (a), (b) or (c) above; or
(f) a composition comprising any one of (a), (b), (c), (d), or (e) above.
   Another aspect provides a method of reducing or preventing retinal ganglion cell degeneration and/or optic nerve head excavation in a subject, comprising administering an effective amount of a mutant neuroserpin or portion thereof or a nucleic acid encoding a mutant neuroserpin protein or portion thereof as described herein.

An alternative aspect provides a mutant neuroserpin or portion thereof or a nucleic acid encoding a mutant neuroserpin protein or portion thereof as described herein for use in reducing or preventing retinal ganglion cell degeneration and/or optic nerve head excavation in a subject; or use of a mutant neuroserpin or portion thereof or a nucleic acid encoding a mutant neuroserpin protein or portion thereof as described herein in the manufacture of a medicament for reducing or preventing retinal ganglion cell degeneration and/or optic nerve head excavation in a subject.

In various embodiments, the subject is administered:
(a) a mutant neuroserpin protein described herein; or
(b) a nucleic acid encoding a mutant neuroserpin protein described herein; or
(c) a vector comprising a nucleic acid encoding a mutant neuroserpin protein described herein; or
(d) a viral particle comprising a vector which comprises a nucleic acid encoding a mutant neuroserpin protein described herein;
(e) a liposome, exosome, nanoparticle, gold particle, or lipoplex, comprising any one of (a), (b) or (c) above; or
(f) a composition comprising any one of (a), (b), (c), (d), or (e) above.

The term “administering” should be understood to mean providing a compound or agent to a subject in need of treatment.

Administration of the agent to subject may be intramuscular, subcutaneous, intraocular, subconjunctival, subretinal, suprachoroidal, intravenous, or intravitreal. Typically, the agent is administered intravitreally.

Delivery to the retinal cells may also be achieved via topical administration such as through eye drops, with enhanced penetration techniques such as liposomes or nanoparticles, subconjunctival injection or intravitreal depot implant. Methods for topical administration are described in, for example, (Alqawlaq S., et al. (2014) Preclinical development and ocular biodistribution of gemini-DNA nanoparticles after intravitreal and topical administration: towards non-invasive glaucoma gene therapy. Nanomedicine, 10, 1637-47; Liaw, J., et al. (2001). In vivo gene delivery into ocular tissues by eye drops of poly(ethylene oxide)-poly(propylene oxide)-poly(ethylene oxide) (PEO-PPO-PEO) polymeric micelles. Gene Ther, 8, 999-1004; Liu, C. et al (2016). Facile Noninvasive Retinal Gene Delivery Enabled by Penetratin. ACS Appl Mater Interfaces, 8, 19256-67).

Compositions suitable for intravenous, subcutaneous, intramuscular, or intravitreal use include sterile aqueous solutions or dispersions and sterile powders for the extemporaneous preparation of sterile injectable solutions or dispersions. The pharmaceutically acceptable carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride.

In embodiments in which the agent is packaged in a viral particle, the pharmaceutical compositions may comprise viral particles in any concentration that allows the agent to be effective. In such embodiments, the pharmaceutical compositions may comprise the virus particle in an amount of from 0.1% to 99.9% by weight. Pharmaceutically acceptable carriers include water, buffered water, saline solutions such as, for example, normal saline or balanced saline solutions such as Hank's or Earle's balanced solutions), glycine, hyaluronic acid etc. Titers of viral particles to be administered will vary depending on, for example, the particular vector to be used, the mode of administration, extent of the condition, the individual, and may be determined by methods standard in the art. The therapeutic and/or pharmaceutical compositions, in some embodiments, may contain viral particles per dose in a range of, for example, from about 10⁴ to about 10¹¹ particles, from about 10⁵ to about 10¹⁰ particles, or from about 10⁶ to about 10⁹ particles. In the context of AAV vectors, vector genomes are provided in in a range of, for example, from about 10⁴ to about 10¹⁴ vector genomes, from about 10⁵ to about 10¹³ vector genomes, from about 10⁶ to about 10¹³ vector genomes, from about 10⁷ to about 10¹³ vector genomes, from about 10⁸ to about 10¹³ vector genomes, or from about 10⁹ to about 10¹³ vector genomes. Such doses/quantities of AAV vector are useful in the methods described herein.

It will be understood that the specific dose level and frequency of dosage for any particular subject may be varied and will depend upon a variety of factors including, for example, the activity of the specific compound or agent employed, the metabolic stability and length of action of that compound or agent, the age, body weight, general health, sex, diet, mode and time of administration, drug combination, the severity of the particular condition, and the host undergoing therapy.

Also provided is a kit, comprising nucleic acid encoding mutant neuroserpin, mutant neuroserpin protein, vector, viral particle, and/or composition described herein. The kit typically comprises a container comprising the nucleic acid encoding mutant neuroserpin, mutant neuroserpin protein, vector, viral particle, and/or composition described herein. The container may be simply a bottle comprising the agent in parenteral dosage form, each dosage form comprising a unit dose of the agent. The kit will typically further comprise printed instructions. In some embodiment, the kit may comprise a label or the like, indicating treatment of a subject according to the present method. In one form, the kit may be a container comprising the agent in a form for parenteral dosage. For example, the agent may be in the form of an injectable solution in a disposable container, or in the form of a solution in an eye dropper container. In some embodiments, the kit may comprise the components for preparing stable transfectants for producing neuroserpin protein. Such a kit may contain transfected cells in a frozen state, or may comprise isolated nucleic acid encoding mutant neuroserpin for transforming or transfecting bacterial or eukaryotic cells for protein production.

Nucleic acid encoding the mutant neuroserpin protein and vectors comprising the nucleic acid encoding the mutant neuroserpin protein described herein may be introduced into host cells in vitro for expression of the mutant neuroserpin or portion thereof in the host cell. Accordingly, one aspect provides a host cell comprising nucleic acid encoding the mutant neuroserpin protein or portion thereof described herein or a vector comprising the nucleic acid encoding the mutant neuroserpin protein or portion thereof described herein. The host cell may be any host cell suitable for expression of the mutant protein. The host cell may be prokaryotic or eukaryotic cell. In one embodiment, the cell is a prokaryotic cell (e.g., E. coli). In one embodiment, the host cell is a eukaryotic cell. In one embodiment, the host cell for in vitro expression of protein is a mammalian cell. Mammalian cells for expression of proteins in include, for example, CHO cells, NSo, Sp2/o, HeLa, HEK, and CAP. In one embodiment, the host cell for in vitro expression of protein is a yeast cell. Examples of yeast cells for in vitro expression of protein include Pichia pastoris and Saccharomyces cerevisiae.

A further aspect provides a method of producing a mutant neuroserpin protein or portion thereof as described herein, comprising incubating a host cell comprising nucleic acid encoding the mutant neuroserpin protein or portion thereof or vector comprising nucleic acid encoding the mutant neuroserpin protein or portion thereof described herein, under conditions which promote expression of the mutant neuroserpin protein or portion thereof. In one embodiment, the method further comprises isolating the mutant neuroserpin protein or portion thereof. Methods of expression and isolation of recombinant protein are known in the art.

As used herein, “% identity” with reference to a polypeptide, or “% identical to the amino acid sequence of a polypeptide”, refers to the percentage of residues in the two sequences that are the same when aligned for maximum correspondence over a specified comparison window, as measured by sequence comparison algorithms or by visual inspection.

Sequence comparison algorithms for determining % identity between two polypeptides are known in the art. Examples of such algorithms are the algorithm of Myers and Miller (1988); the local homology algorithm of Smith et al. (1981); the homology alignment algorithm of Needleman and Wunsch (1970); the search-for-similarity-method of Pearson and Lipman (1988); the algorithm of Karlin and Altschul (1990), modified as in Karlin and Altschul (1993). Computer implementations of these algorithms for determining % identity between two polypeptides include, for example: CLUSTAL (available from Intelligenetics, Mountain View, Calif.) (Pearson et al. (1994)).; the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA).

As used herein, a “subject” is a mammal. The mammal can be a human or a non-human. Examples of non-humans include non-human primate, sheep, mouse, rat, dog, cat, horse, cow, pig, or any other mammals which can suffer from glaucoma. Typically, the subject is a human.

The term “effective amount” refers to the amount of the compound that will elicit the biological or medical response of a tissue, system, animal or human that is being sought by the researcher, veterinarian, medical doctor or other clinician.

As used herein, “treating” means affecting a subject, tissue or cell to obtain a desired pharmacological and/or physiological effect and includes inhibiting the condition, i.e. arresting its development; or relieving or ameliorating the effects of the condition i.e. cause reversal or regression of the effects of the condition.

As used herein, “preventing” means preventing a condition from occurring in a cell or subject that may be at risk of having the condition, but does not necessarily mean that condition will not eventually develop, or that a subject will not eventually develop a condition. Preventing includes delaying the onset of a condition in a cell or subject.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In order to exemplify the nature of the present invention such that it may be more clearly understood, the following non-limiting examples are provided.

Examples

WT and mutant neuroserpin coding mRNA were cloned separately into the pSF-CAG-GFP plasmid and used to transfect SHSY5Y cells. The human neuroserpin amino acid sequence is provided in FIG. 1A. Mutant neuroserpin was prepared by specifically mutating wild-type neuroserpin at Met 363 position to introduce an Arg residue. The plasmid map of WT, mutant and only eGFP coding vectors is shown in FIG. 1B, 1C and 1D.

The SH-SY5Y neuronal cells (ATCC, USA) were grown in DMEM media supplemented with 10% fetal bovine serum (FBS), penicillin (100 U/ml), streptomycin (100 U/ml) and 2 mM L-glutamine. The cells were kept at 37° C. in a humidified chamber containing 5% CO₂. Approximately, 2.0×10⁵ cells were seeded and grown to 80% confluency prior to transfection with pSF-CAG-GFP plasmids using lipofectamine treatment. Cells were pre-differentiated with 10 μM all-trans retinoic acid (Sigma) for 2 days. Medium was changed into retinoic acid medium without antibiotics and cells transfected. The cells were fixed in 4% paraformadehyde for 10 minutes and rinsed with phosphate buffered saline (PBS) several times. The cells were blocked with PBS containing 5% normal serum and 0.3% Triton X-100 for 1 h and subsequently treated with primary antibodies against either neuroserpin (1:100) or GFP (1:100) overnight at 4° C. followed by one hour incubation (1 hour) with the secondary antibodies linked to fluorescent probes. Images were acquired using a Zeiss fluorescence microscope. The results showed that both WT and mutant form of neuroserpin were well expressed in the SHSY5Y cells following transfection. The expression was also confirmed using GFP staining with GFP specific antibodies.

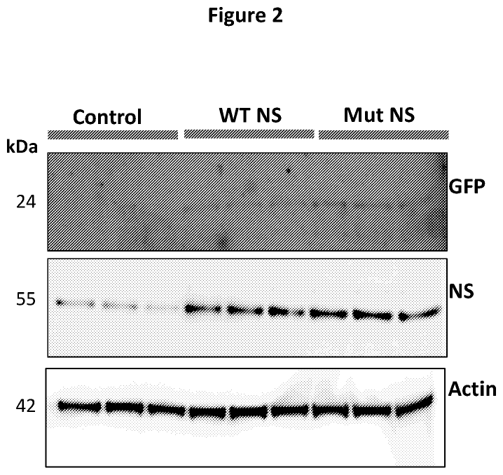

The expression of WT and mutant neuroserpin proteins was also investigated using western blotting. Briefly, the cells were lysed in buffer (20 mM HEPES, pH 7.4, 1% Triton X-100, 1 mM EDTA) containing 10 μm/ml aprotinin, 10 μM leupeptin, 1 mM PMSF and 1 mM NaVO₃, 100 mM NaF, 1 mM Na₂MoO₄ and 10 mM Na₄P₂O₇. The proteins were resolved in SDS-PAGE and transferred to PVDF membranes. Membranes were blocked in Tris-buffered saline (TTBS) (20 mM Tris-HCl [pH 7.4], 100 mM NaCl, and 0.1% Tween 20) containing 5% skimmed milk and incubated overnight with anti-GFP (1:1000) and anti-neuroserpin (1:1000) antibodies at 4° C. and horseradish peroxidase (HRP)-linked secondary antibodies for 1 hour. After extensive washing, antibody detection was accomplished with Supersignal West Pico Chemiluminescent substrate (Pierce). Signals were detected using an automated luminescent image analyzer (ImageQuant LAS 4000, GE Healthcare). The western blot results support the immunofluorescence findings and demonstrate that both WT and mutant form of neuroserpin were well expressed in the transfected cells compared to the control cells (FIG. 2).

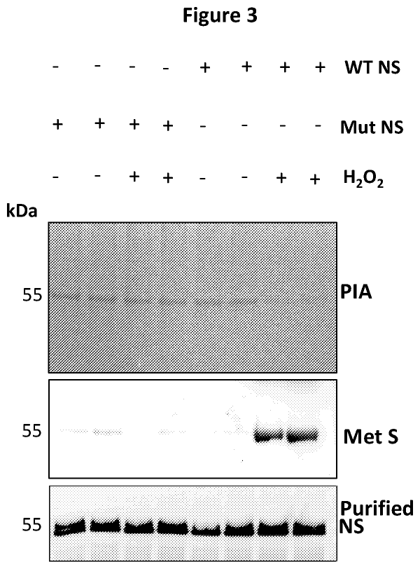

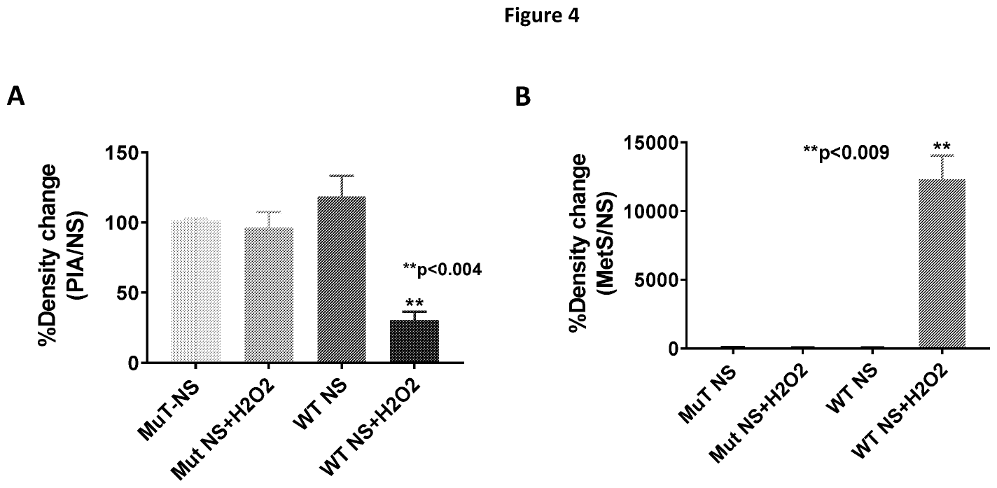

The WT and mutant form of neuroserpin were purified from the SHSY5Y cell lysates. WT and mutant forms of neuroserpin (1 μg/μL) were separately incubated with H₂O₂ (10 μM for 1 hour) in microtubes for oxidation at room temperature. The proteins were subjected to western blotting as shown in FIG. 3. The blots were probed with neuroserpin and methionine sulfoxide (MetS) antibodies. Methionine is converted to MetS upon oxidation and MetS reactivity reflects the oxidation of this amino acid. The experiment showed that while WT neuroserpin had significantly increased MetS reactivity following incubation with H2O₂, the MetS reactivity in mutant neuroserpin was not altered (FIG. 4B). Further, we also analysed the plasmin inhibitory activity (PIA) of WT and mutant forms of neuroserpin with and without incubation with H₂O₂. The PIA of the protein was studied using gelatin gel zymography (FIG. 4). The PIA was significantly reduced in the case of WT neuroserpin that was incubated with H₂O₂. In contrast, the PIA activity of mutant neuroserpin was not altered to any significant extent (FIG. 4A). This experiment indicates that mutant neuroserpin is more resistant to oxidative stress conditions compared to the natural WT neuroserpin molecule.

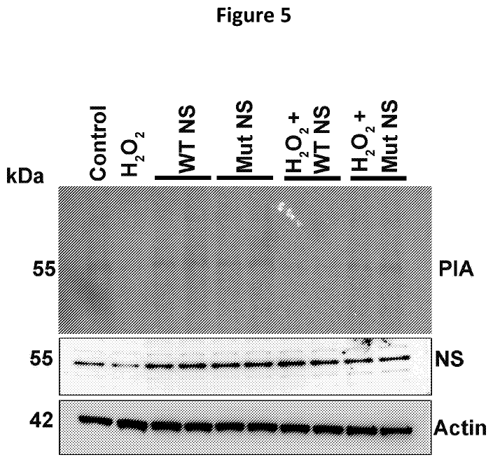

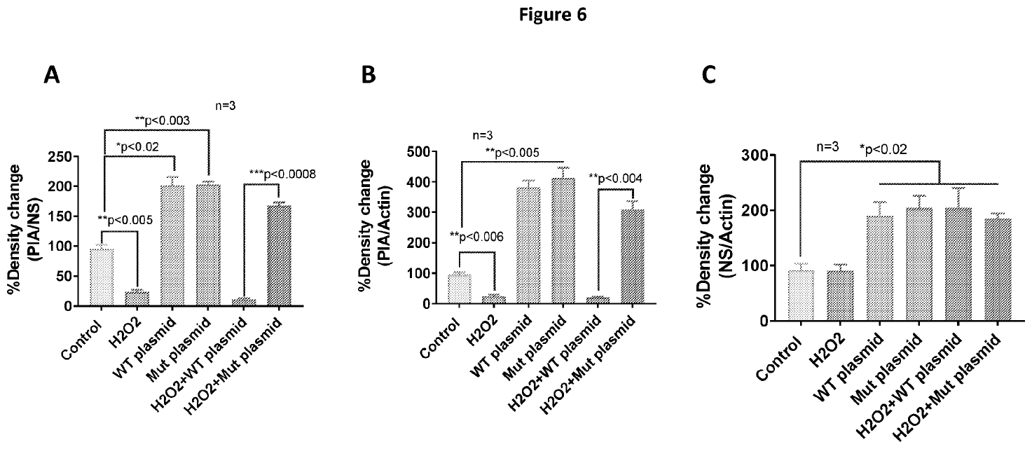

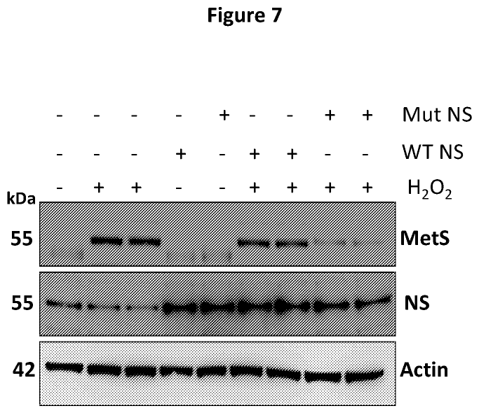

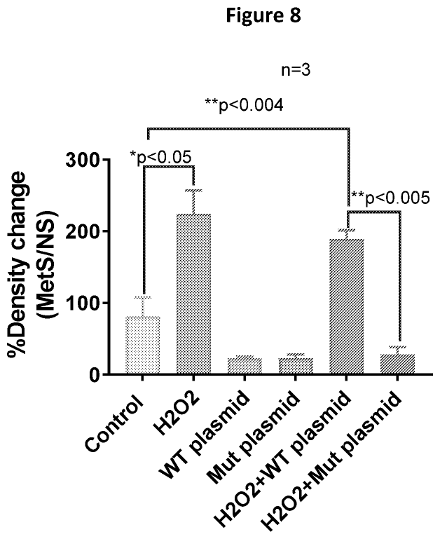

The susceptibility of WT and mutant forms of neuroserpin to oxidative stress was also investigated using an alternative approach where the SHSY5Y cells expressing neuroserpin were directly exposed to H₂O₂ treatment (10 μM, 6 hrs). The cell lysates were subjected to western blotting and subsequently blots probed with neuroserpin and actin antibodies. The cell lysates were also subjected to PIA analysis using gelatin gel zymography (FIG. 5). The band intensities were quantified using densitometric analysis. Results indicate that PIA of the WT neuroserpin was significantly decreased following H₂O₂ treatment while the PIA of mutant neuroserpin was preserved even after H₂O₂ treatment. The PIA activity was compared both with respect to the total neuroserpin protein expression as well as to that of the total actin (FIG. 6A, B). The total neuroserpin protein expression was comparable between the WT and mutant plasmid transfection groups with and without the H₂O₂ treatment (FIG. 6C). The changes in MetS reactivity of WT and mutant neuroserpin in the cells was also investigated in the cell lysates following western blotting (FIG. 7). The experiment revealed that cells subjected to H₂O₂ treatment had significantly increased MetS reactivity compared to the control cells. Further, the WT neuroserpin had significantly more MetS reactivity compared to the mutant neuroserpin following H₂O₂ treatment (FIG. 8). This experiment demonstrated that mutant form of neuroserpin is more resistant to oxidative stress compared to the WT form of neuroserpin protein.

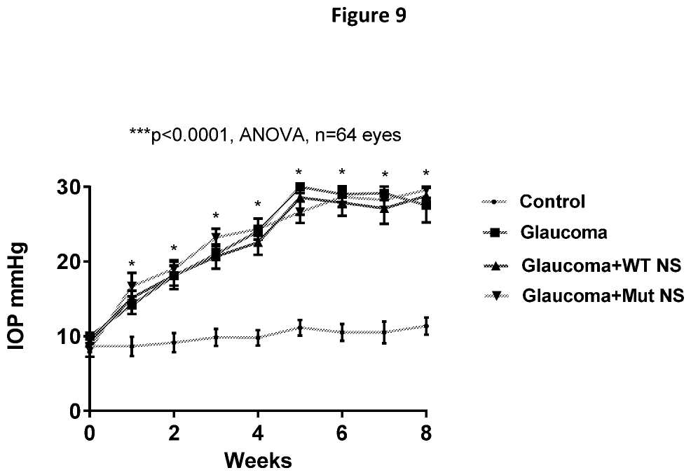

Literature evidence indicates that neuroserpin is well expressed in the retina and undergoes oxidative inactivation in glaucoma conditions. Therefore, we sought to investigate whether neuroserpin administration in experimental model of glaucoma can impart protection to the retina. In order to study this, we generated a mouse model of chronic glaucoma by repeated microbead injections of 10 μm fluorospheres (Fluorospheres, 10 μM). Weekly injections into the anterior chamber of eye resulted in sustained increase in IOP for 2 months (control 10.5±1.2; Glaucoma 24.6±1.3 mmHg) following which the tissues were harvested for further biochemical analysis (FIG. 9).

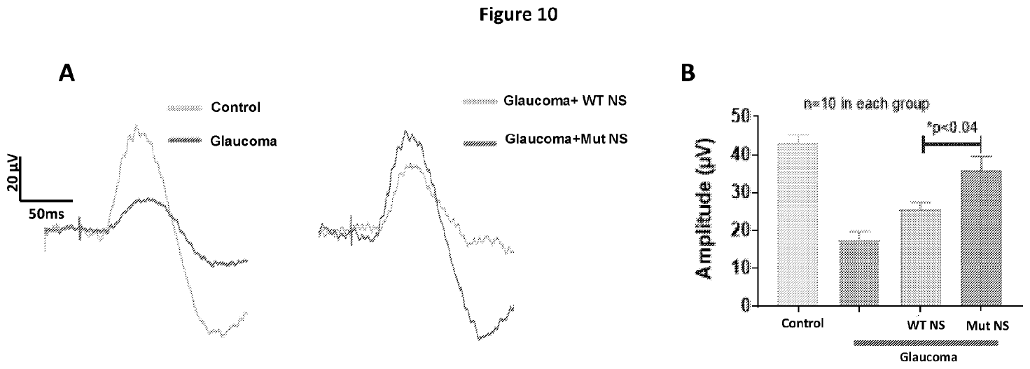

Electroretinographic (ERG) recordings were performed in control and glaucoma mice as well as in the animals that were subjected to either WT or mutant neuroserpin treatment (FIG. 10A). Neuroserpin was administered to the mice through weekly intravitreal injections under anaesthesia (1 μg/μL, 2 μL vol). For electrophysiological recordings, the animals were dark-adapted overnight and anaesthetised with ketamine and medetomidine (75 and 0.5 mg/kg, respectively), and pupils were dilated using 2.5% phenylephrine, after which 1% tropicamide and topical anaesthetic (1% proparacaine) were applied to the cornea. Positive scotopic threshold response (pSTR) recordings, which are a measurement of inner retinal function were recorded using flash intensities of −4.3 log cd·s/m² delivered 30 times at a frequency of 0.5 Hz. The pSTR amplitudes were measured from baseline to the positive peak observed around 120 ms. Quantification showed that there was significant reduction of pSTR amplitudes in animals subjected to increased IOP. The retinas were protected against the glaucoma damage in both WT and mutant neuroserpin treated animals. The mutant neuroserpin treated group however, demonstrated a significant more protection compared to both glaucoma alone as well as glaucoma+WT neuroserpin treated mice (FIG. 10B).

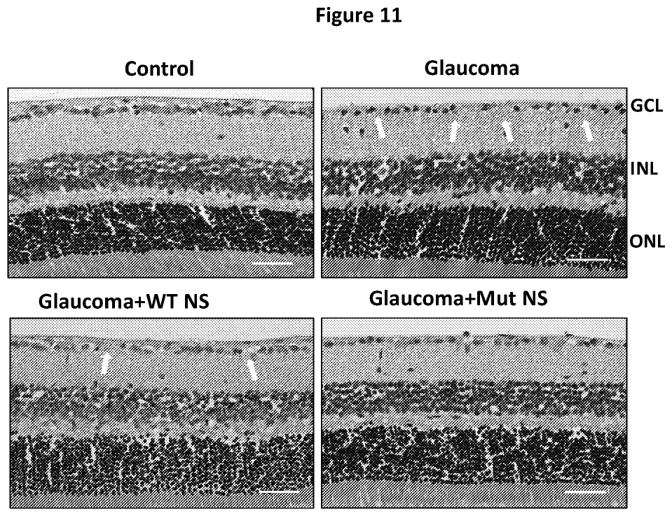

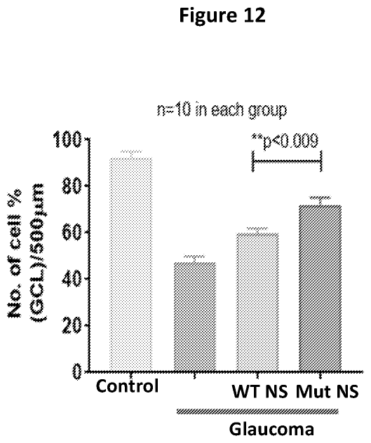

The protective effects of mutant neuroserpin administration on the retinal structure were further investigated using histological analysis. The animals were sacrificed and perfused transcardially with 4% paraformaldehyde. Eyes were harvested, fixed in 4% (w/v) paraformaldehyde, processed in an automatic tissue processor (Leica, Germany), and embedded in paraffin. Care was taken to ensure that the orientations of the eyes were identical by using tissue marking dye and 5-μm thick sagittal sections of the eye were made using a rotary microtome (Carl Zeiss, Germany). Tissues were mounted and subjected to H and E staining (FIG. 11). Cell density in the GCL was quantified for each eye by counting the number of cells in the GCL over a distance of 500 μm from the edge of the optic disc for both superior and inferior retina (FIG. 12). Both WT and mutant neuroserpin administered groups showed protection of the GCL against glaucoma damage. The mutant neuroserpin treated group however showed a significantly greater number of cells in the GCL compared to both glaucoma alone and glaucoma+WT neuroserpin treated mice eyes.

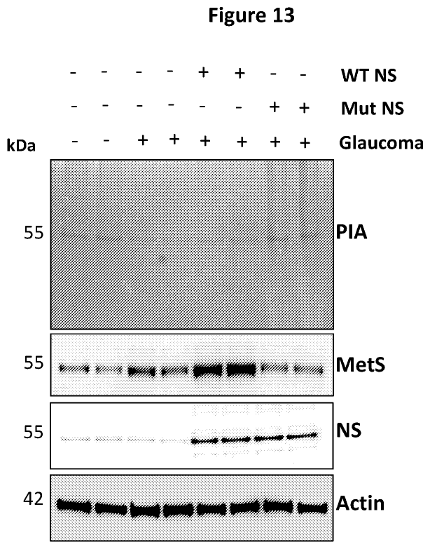

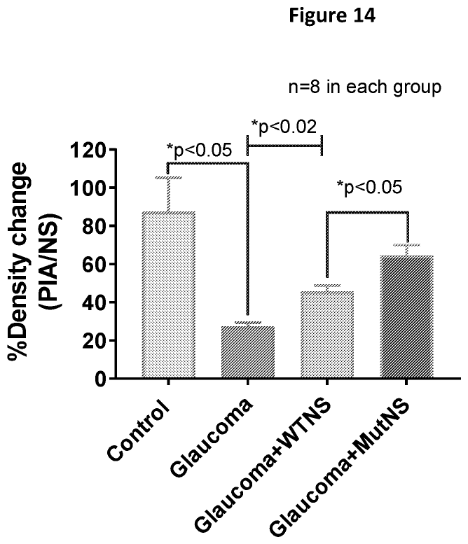

The retinas were also harvested for biochemical analysis and analysed using western blotting. The retinas were lysed in lysis buffer (2 mM HEPES, pH 7.4, 1% Triton X-100, 1 m EDTA) containing 10 μg/ml aprotinin, 10 μM leupeptin, 1 mM PMSF and 1 mM NaVO₃, 100 mM NaF, 1 mM Na₂MoO₄ and 10 mM Na₄P₂O₇ and proteins resolved by 10% SDS-PAGE and transferred to PVDF membranes. The blots were probed using specific antibodies against neuroserpin, MetS and actin. Additionally, the proteins were subjected to native gelatin gel electrophoresis under non-denaturing conditions to assess PIA using gelatin gel zymography (FIG. 13). A much higher MetS reactivity was observed in the mice retinas subjected to experimental glaucoma paradigm. The MetS reactivity was more in the WT neuroserpin administered mice group. The mutant neuroserpin administered mice however showed much lower levels of MetS reactivity. Comparable levels of neuroserpin were detected in the retinal lysates of WT and mutant neuroserpin administered mice. Actin blots demonstrated that equal quantities of protein was loaded in each case. The densitometric quantification of PIA band intensity compared to the total neuroserpin levels indicated that while the PIA activity was much reduced in glaucoma conditions, it was rescued to significant extent in the WT and mutant neuroserpin administered groups. Further, the mutant neuroserpin treated group demonstrated a much higher PIA compared to the WT neuroserpin treated mice (FIG. 14). This experiment demonstrates that mutant neuroserpin is able to better retain its PIA when compared to the WT neuroserpin under the glaucomatous stress conditions.

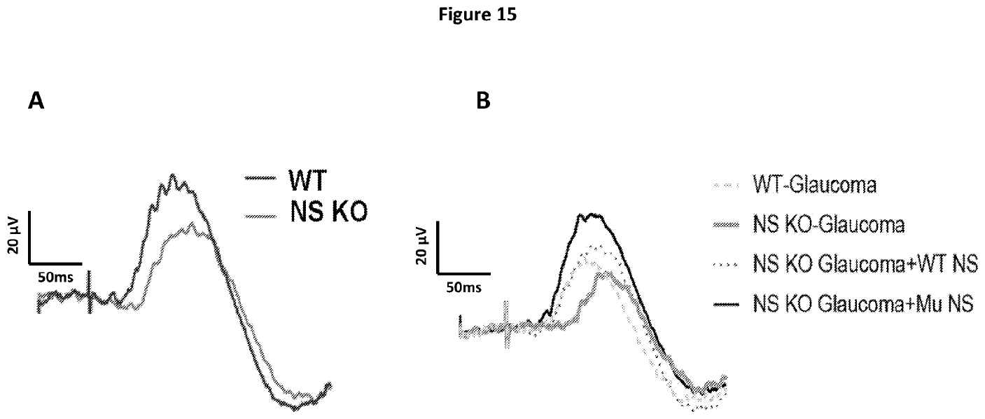

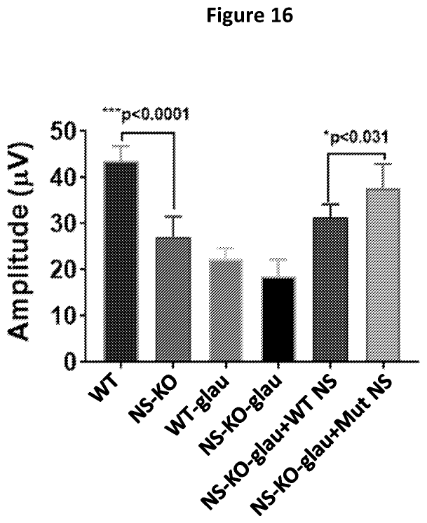

The protective role of neuroserpin in the retina was further established in a neuroserpin knockout mouse model. These animals are global knockout and genetically deficient in neuroserpin starting from birth. Retinal electrophysiological recordings from these animals showed reduced pSTR amplitudes (FIG. 15A). This indicated that neuroserpin plays an important role in maintenance of inner retinal function. We further subjected the WT and neuroserpin knockout animals to chronic glaucoma conditions using anterior chamber microbead injections (weekly injections, 2 months). The neuroserpin knockout mice were administered neuroserpin protein (WT or mutant neuroserpin) through intravitreal injections (1 μg/μL; 2 μL vol. weekly injections, 2 months). Quantification of pSTR amplitudes revealed that both WT and neuroserpin administered neuroserpin knockout mice were significantly protected against the glaucoma damage (FIG. 15B). However, the mutant neuroserpin treated mice showed a greater protection as compared to the WT neuroserpin (FIG. 16).

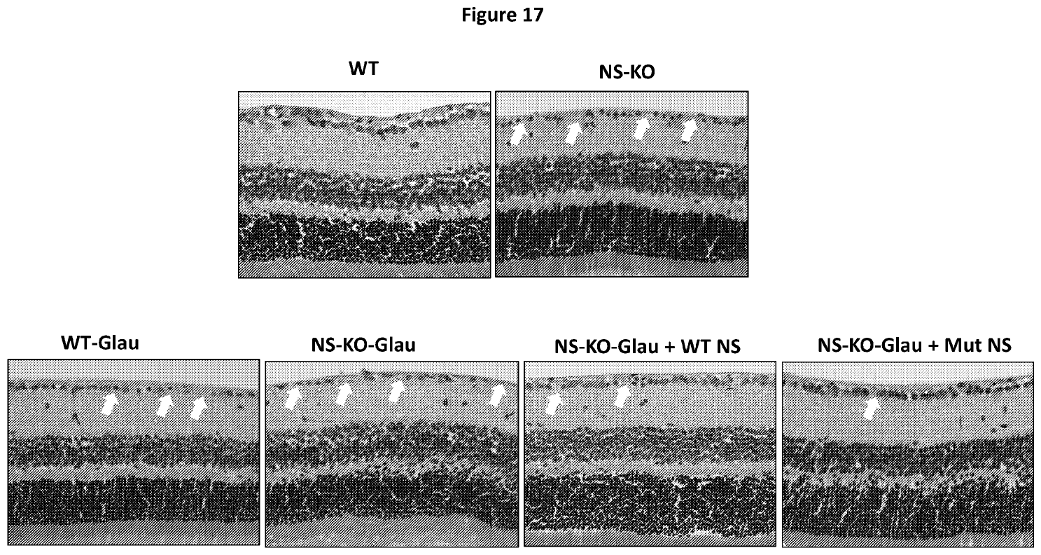

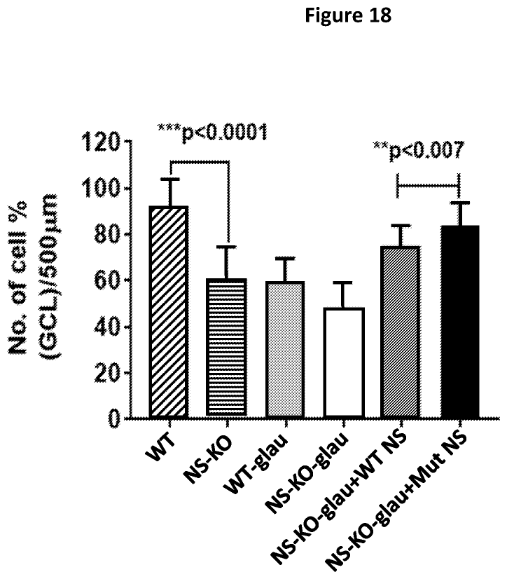

The neuroserpin knockout mice retinas were further evaluated by histological analysis utilising H and E staining. A thinning of the ganglion cell layer was observed in the neuroserpin knockout animals compared to the WT mice. When the neuroserpin knockout model was superimposed with chronic glaucoma model (increased IOP using microbead injections), there was further thinning of the GCL. The neuroserpin knockout mice were further subjected to intravitreal neuroserpin administration (WT or mutant neuroserpin; 1 μg/μL; 2 μL vol. weekly injections, 2 months). Histological analysis revealed that both WT and mutant neuroserpin administered animals showed a protection against GCL loss (FIG. 17). Quantification of the data showed that the mutant neuroserpin administration conferred a much greater protection when compared to the WT neuroserpin in mice (FIG. 18). These results establish that mutant neuroserpin administration can rescue the neuroserpin knockout retinal phenotype and can impart much higher protection compared to the WT native neuroserpin protein.

AAV Mediated Overexpression of Neuroserpin in RGCs

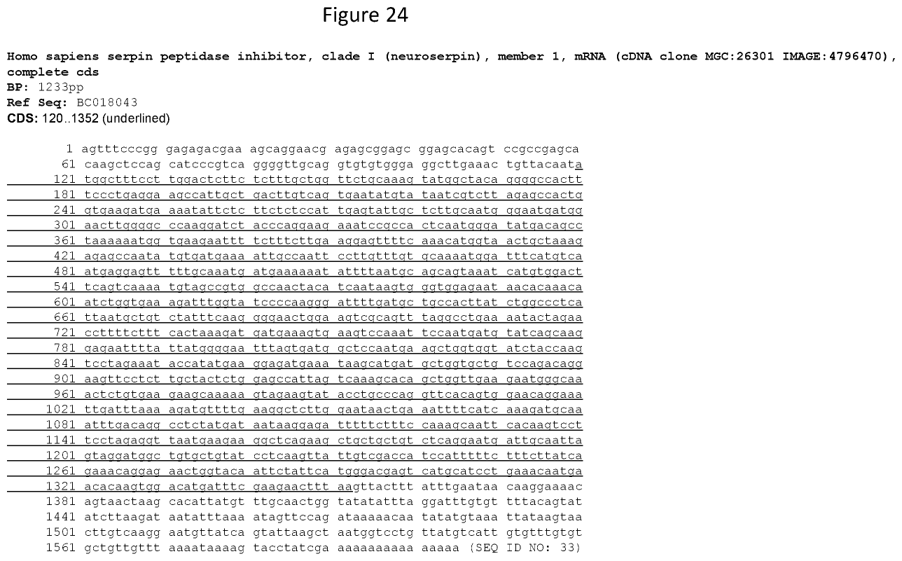

Adeno-associated virus backbone, serotype 2 (AAV2) vectors were produced commercially by Vector Biolabs (293 Great Valley Parkway, Malvern, PA19355, USA). Briefly, the human neuroserpin (NS) cDNA (BC018043-FIG. 24) was placed under the modified transcriptional control of the cytomegalovirus (CMV) and chicken β-actin rabbit beta-globin (CBA) known as CAG2 hybrid promoter, shortened Woodchuck Hepatitis Virus Posttranscriptional Regulatory Element (WPRE), polyadenylation signal (polyA) and inserted into the AAV2 backbone, green fluorescence protein (eGFP) vector (AAV2-CAG2-eGFP-WPRE) and NS over expression (AAV2-CAG2-eGFP-T2A-hNS-WPRE or AAV2-NS). Enhanced green fluorescent protein (eGFP) and NS gene sequences were driven by CAG2 hybrid promoter including 2A linker. eGFP control vector was also expressed under the control CAG promoter flanked by AAV terminal repeats (briefly referred to AAV-GFP) and used as a control for NS overexpression.

Animal eyes were injected with AAV2 constructs comprising either green fluorescence protein (AAV2-GFP) or GFP tagged with mutant neuroserpin M363R (AAV2-NS). GFP alone was used as control (final concentration, 5.8×10¹ GC/mL and 6.5×10¹ GC/mL for AAV-GFP and AAV-NS respectively). Expression of GFP was observed throughout the retina from both viral vectors, indicating that the expressed protein could be delivered effectively to the retina using AAV delivery.

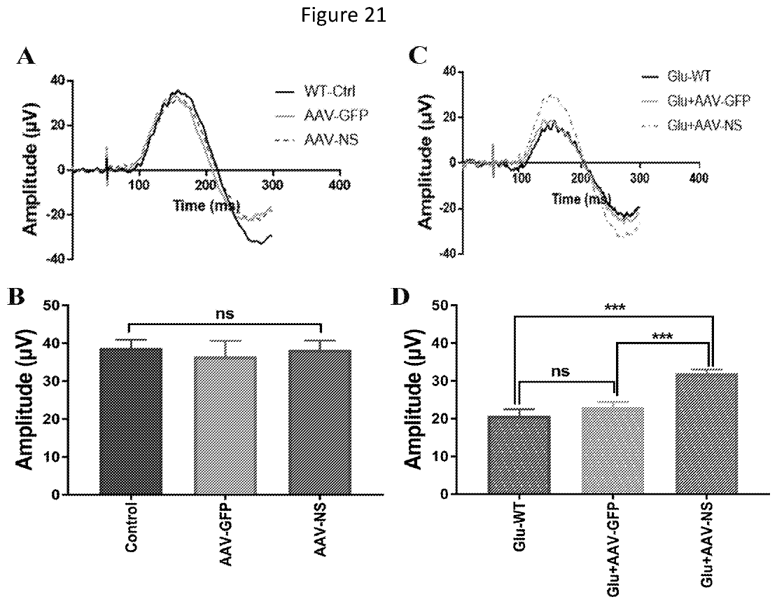

AAV mediated overexpression of neuroserpin was used to rescue retinal function in an experimentally induced high intraocular pressure (TOP) induced mouse model. High IOP was induced in mice as described above. Healthy and high TOP induced mice were then administered AAV2-GFP and AAV2-NS. pSTR responses in WT mice in a healthy condition treated with either AAV2-GFP (green) or AAV2-NS (pink) was compared with non-treated control (blue) retinas. The results are shown in FIG. 21A and B. No change in the pSTR amplitude was observed between non-treated and AAV treated animal in healthy condition (n=10 animals in each group). High IOP mice (experimental glaucoma) were also treated with AAV2-GFP and AAV2-NS, and the results are shown in FIGS. 21C and D. pSTR responses from experimental glaucoma (light blue), glaucoma+AAV2-GFP (green) and glaucoma+AAV2-NS (pink) treated mice showed a significant decline in p-STR amplitude in both high IOP alone or animal expressing GFP in high IOP compared to control after 8 weeks of multiple microbead injections. AAV mediated overexpression of neuroserpin significantly protect the pSTR amplitude in experimental glaucoma (n=10 animals in each group, ***p<0.0004).

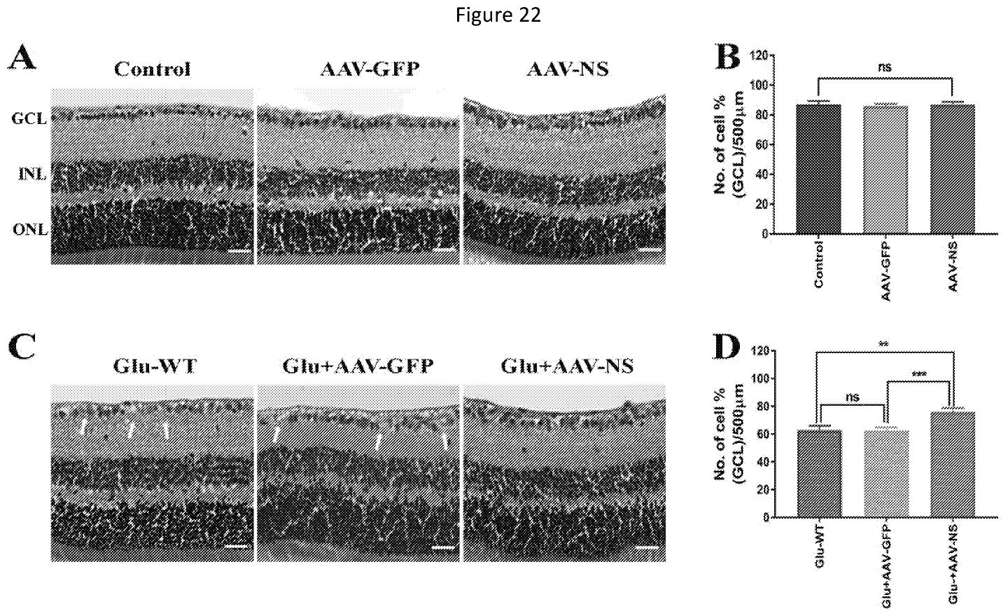

Healthy mice treated with either AAV2-GFP (green) or AAV2-NS (pink) was compared with non-treated control (blue) retinas. Tissue sections of retinas showed no change between the different treatments (FIGS. 22A and B). Glaucoma induced mice treated with either AAV2-GFP (green) or AAV2-NS (pink) was compared with non-treated control (blue) retinas. The results are shown in FIGS. 22C and D. Non-treated mice and mice treated with AAV2-GFP showed a statistically significant reduction in retinal cells compared to mice treated with AAV2-NS.

These results show that that mutant neuroserpin delivered using AAV effectively rescues retinal function in an experimental animal model of glaucoma.

In Silico Analysis

An analysis of hydrogen bonds and salt bridges was performed on a mutant human neuroserpin comprising a single M363R mutation using molecular dynamics. All systems for molecular dynamics (MD) simulations were prepared by soaking in a cubic box filled with TIP3P water molecules. Simulation systems were neutralized by adding Na+ or Cl−ions. MD runs were carried out at constant temperature of 300 K for 500 ns using Gromacs version 5.0.4. A time step of 2 fs was used for the simulations. A cut-off of 10 Å was used for short range interactions while Particle-Mesh Ewald (PME) method was used to handle long-range interactions. Coordinates and velocities were saved every 10 ps.

Molecular dynamics trajectories were processed and analyzed using MDAnalysis, MDTraj and scikit-learn python libraries. Contacts between wildtype and mutant Neuroserpin and tPA were analyzed using GetContacts program. Contacts were visualized using Flareplot. All graphics were prepared using PyMOL and R environment for statistical computing.

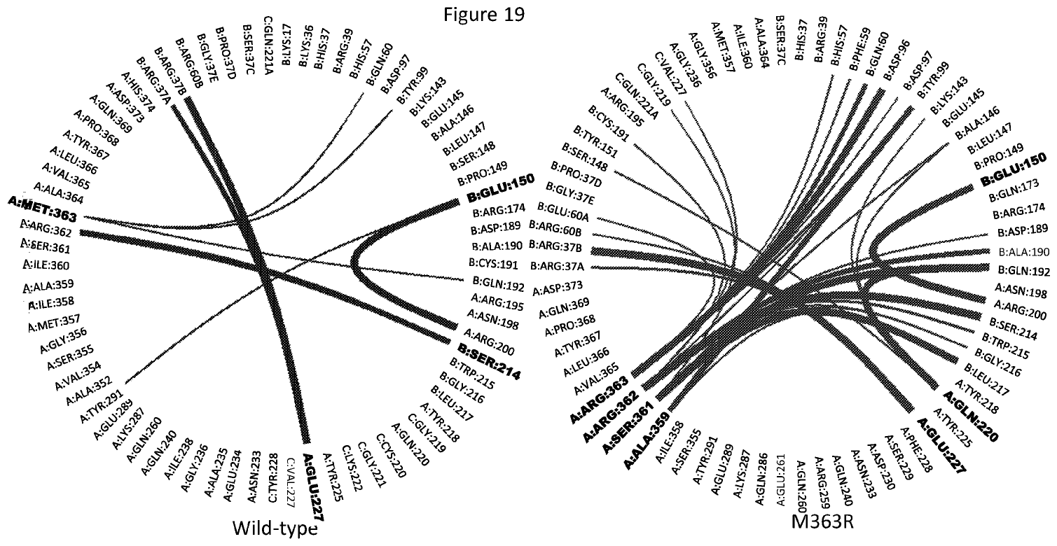

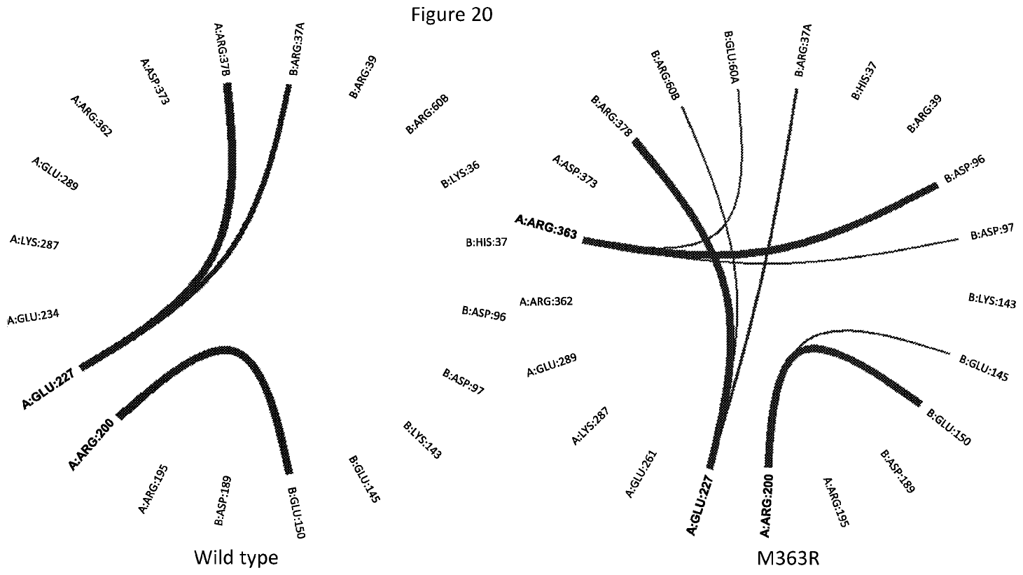

The results of the hydrogen bond analysis on wild type and mutant M363R is shown in FIG. 19. The results of salt bridge analysis is shown in FIG. 20.

CUPSAT—(Cologne University Protein Stability Analysis Tool) was used to study the protein stability upon point mutation using alternative amino acids to arginine. It was determined that in addition to arginine, substitution of methionine at position 363 of neuroserpin with the amino acids leucine, serine, threonine, asparagine, glutamic acid, and aspartic acid also had a stabilizing effect on the molecule.

1. A nucleic acid comprising a nucleotide sequence encoding a mutant neuroserpin protein or a portion thereof, the mutant neuroserpin or portion thereof comprising an amino acid sequence that is at least 60% identical to the amino acid sequence of SEQ ID NO : 2, and which comprises a reactive centre loop comprising the amino acid sequence MIAX₃SRX₁X₂VL, wherein:
X₁ is selected from R, S, T, F, N, E, K, and D;
X₂ is M or A; and
X₃ is I, V or N,
and wherein the mutant neuroserpin protein or portion thereof has serine protease inhibition activity and is at least partially resistant to oxidative inactivation.

2. The nucleic acid of claim 1, wherein the mutant neuroserpin or portion thereof comprises a reactive centre loop comprising the amino acid sequence MIAISRX₁AVL, wherein X₁ is selected from R, S, T, F, N, E, K, and D.

3. A nucleic acid comprising a nucleotide sequence encoding a mutant neuroserpin protein or a portion thereof, the mutant neuroserpin or portion thereof comprising an amino acid sequence which differs from the corresponding wild-type neuroserpin in at least a substitution of methionine in the amino acid sequence IAISRMAVL of the wild-type neuroserpin, wherein the mutant neuroserpin protein or portion thereof has serine protease inhibition activity and is at least partially resistant to oxidative inactivation.

4. The nucleic acid of claim 3, wherein the mutant neuroserpin protein or a portion thereof comprises the amino acid sequence IAISRX₁AVL, wherein X₁ is an amino acid other than M.

5. The nucleic acid of claim 4, wherein X₁ selected from R, S, T, F, N, E, K, and D.

6. The nucleic acid of any one of claims 1 to 5, wherein the serine protease inhibition activity comprises inhibition of plasmin, tissue plasminogen activator and/or urokinase plasminogen activator.

7. The nucleic acid of any one of claim 1, 2, 4 or 5, wherein X₁ is R.

8. The nucleic acid of any one of claims 3 to 7, wherein the amino acid substitution is M363R.

9. The nucleic acid of any one of claims 1 to 8, wherein the mutant neuroserpin comprises the amino acid sequence of SEQ ID NO: 2.

10. The nucleic acid of any one of claims 1 to 9, wherein the mutant neuroserpin consists essentially of the amino acid sequence of SEQ ID NO: 2.

11. The nucleic acid of any one of claims 1 to 10, comprising a regulatory sequence operatively linked to the nucleotide sequence encoding mutant neuroserpin for permitting expression of the mutant neuroserpin in a host cell.

12. The nucleic acid of claim 11, wherein the host cell is an RGC.

13. The nucleic acid of claim 11 or 12, wherein the regulatory sequence comprises a promoter.

14. The nucleic acid of claim 13, wherein the promoter is RCG specific promoter.

15. A vector comprising the nucleic acid of any one of claims 1 to 14.

16. The vector of claim 15, wherein the vector is a viral vector.

17. The viral vector of claim 16, wherein the viral vector is selected from adenovirus and adeno-associated virus (AAV).

18. The viral vector of claim 17, wherein the AAV vector comprises a VP1, VP2 and/or VP3 capsid sequence having at least 90% identity to the VP1, VP2 and/or VP3 sequences of AAV1, AAV2, AAV3, AAV4, AAV5, AAV6, AAV7, AAV8, AAV9, AAV10, AAV11, Rh10, Rh74 or AAV-2i8.

19. The vector of any one of claims 15 to 18, comprising a poly-A sequence located 3′ of the nucleotide sequence encoding mutant neurotrophin.

20. The vector of any one of claims 15 to 19, further comprising one or more AAV inverted terminal repeat (ITR) sequences.

21. A mutant neuroserpin or portion thereof comprising an amino acid sequence that is at least 60% identical to the amino acid sequence of SEQ ID NO: 2, and which comprises a reactive centre loop comprising the amino acid sequence MIAX₃SRX₁X₂VL, wherein:
X₁ is selected from R, S, T, F, N, E, K, and D;
X₂ is M or A; and
X₃ is I, V or N,
and wherein the mutant neuroserpin protein or portion thereof has serine protease inhibition activity and is at least partially resistant to oxidative inactivation.

22. The mutant neuroserpin or portion thereof of claim 19, which comprises a reactive centre loop comprising the amino acid sequence MIAISRX₁AVL, wherein X₁ is selected from R, S, T, F, N, E, K, and D.

23. A mutant neuroserpin protein or a portion thereof, the mutant neuroserpin or portion thereof comprising an amino acid sequence which differs from the corresponding wild-type neuroserpin in at least a substitution of methionine in the amino acid sequence IAISRMAVL of the wild-type neuroserpin, wherein the mutant neuroserpin protein or portion thereof has serine protease inhibition activity and is at least partially resistant to oxidative inactivation.

24. The mutant neuroserpin or portion thereof of claim 3, wherein the mutant neuroserpin protein or a portion thereof comprises the amino acid sequence IAISRX₁AVL, wherein X₁ is an amino acid other than M.

25. The mutant neuroserpin or portion thereof of claim 4, wherein X₁ selected from R, S, T, F, N, E, K, and D.

26. The mutant neuroserpin or portion thereof of any one of claims 21 to 25, wherein the has serine protease inhibition activity comprises inhibition of plasmin, tissue plasminogen activator and/or urokinase plasminogen activator.

27. The mutant neuroserpin or portion thereof of any one of claim 21, 22, 24 or 25, wherein X₁ is R.

28. The mutant neuroserpin or portion thereof of any one of claims 23 to 27, wherein the amino acid substitution is M363R.

29. The mutant neuroserpin or portion thereof of any one of claims 21 to 28, wherein the mutant neuroserpin comprises the amino acid sequence of SEQ ID NO: 2.

30. The mutant neuroserpin or portion thereof of any one of claims 21 to 29, wherein the mutant neuroserpin consists essentially of the amino acid sequence of SEQ ID NO: 2.

31. A viral particle comprising the viral vector of any one of claims 15-20.

32. A pharmaceutical composition comprising the nucleic acid of any one of claims 1-14, the vector of any one of claims 15-20, the viral particle of claim 31, or the mutant neuroserpin protein or portion thereof of any one of claims 21-30.

33. A method of treating or preventing a condition associated with elevated plasmin activity in a subject, comprising administering an effective amount of the nucleic acid of any one of claims 1-14, the vector of any one of claims 15-20, the viral particle of claim 31, the mutant neuroserpin protein or portion thereof of any one of claims 21-30, or the composition of claim 32.

34. The method of claim 33, wherein the nucleic acid is expressed in RGCs of the subject.

35. The method of claim 33 or 34, wherein the condition is glaucoma.

36. A method of reducing or preventing retinal ganglion cell degeneration and/or optic nerve head excavation in a subject, comprising administering an effective amount of the nucleic acid of any one of claims 1-14, the vector of any one of claims 15-20, the viral particle of claim 31, the mutant neuroserpin protein or portion thereof of any one of claims 21-30, or the composition of claim 32.

37. A host cell comprising the vector of claim 15.
